(19) United States
(12) Patent Application Publication
(10) Pub. No.: US 2003/0046716 A1
(43) Pub. Date: Mar. 6, 2003

(54) Transgenically produced platelet derived growth factor

(76) Inventors: Yann Echelard, Jamaica Plains, MA (US); Harry M. Meade, Newton, MA (US); Wolfram Eichner, Butzbach (DE); Klaus Sommermeyer, Rosbach (DE)

Correspondence Address: LOUIS MYERS, Fish & Richardson P.C., 225 Franklin Street, Boston, MA, 02110-2804, US

(21) Appl. No.: 09/884,586

(22) Filed: Jun. 19, 2001

Related U.S. Application Data
(60) Provisional application No. 60/212,406, filed on Jun. 19, 2000.

(51) Int. Cl. A01K 67/027; C07H 21/04; C12N 5/06

(52) U.S. Cl. 800/7; 536/23.5; 435/455; 435/320.1; 435/325

(57) ABSTRACT

The invention features transgenically produced PDGF, e.g., transgenically produced PDGF which is expressed in the milk of a transgenic mammal, and is present in the milk in active form, e.g., as a dimer. The invention also features methods of producing transgenic PDGF, transgenic animals capable of expressing PDGF, and nucleic acid sequences encoding PDGF, e.g., nucleic acid sequences encoding PDGF under the control of a mammary gland specific promoter.

This application claims the benefit of a previously filed Provisional Application No. 60/212,406, filed Jun. 19, 200, the contents of which is incorporated in its entirety.

### BACKGROUND OF THE INVENTION

Growth factors are polypeptide, hormone-like molecules, which interact with specific receptors. They can be present in nanogram amounts in tissue in which a wound healing process can be observed. In fact, the wound healing process is controlled and regulated by growth factors which

(a) have mitogenic activities, which in turn stimulate cellular proliferation;

(b) have angiogenic activities and thus stimulate in growth of new blood vessels;

(c) have chemotactic activities attracting inflammatory cells and fibroblasts to the wound;

(d) influence the synthesis of cytokines and growth factors by neighboring cells;

(e) effect production and degradation of the extracellular matrix.

Platelet-derived growth factor (hereinafter designated PDGF) is a major mitogenic growth factor present in serum but absent in plasma (Antoniades et al., Proc. Nat'l Acad. Sci. USA, vol. 72 (1975), 2635-2639; and Ross and Vogel, Cell, vol. 14 (1978), 203-210). It was discovered upon the observation that serum is superior to plasma in stimulating the in vitro proliferation of fibroblasts (Balk et al., Proc. Nat'l Acad. Sci. USA, vol. 70 (1973), 675-679). PDGF is a mitogen for connective tissue cells as well as most mesenchymally derived cells (Pierce and Mustoe, Annual Review of Medicine, vol. 46 (1995), 467-481) and also acts as a chemotactic factor for neutrophils, monocytes and fibroblasts (Lepisto et al., Eur. Surg. Res., vol. 26 (1994), 267-272). Circulating monocytes and fibroblasts, which migrate into a wound due to chemotactic activity of PDGF, mature to tissue macrophages and are themselves able to secrete PDGF. Besides the chemotactic effect, it has been shown that PDGF-BB induces the expression of tissue factor, the initiator of the clotting cascade, in human peripheral blood monocytes (Ernofsson M., and Siegbahn, A., Thromb. Res., vol. 83 (1996), 307-320).

PDGF also mediates the induction of extracellular matrix synthesis, including production of hyaluronic acid and fibronectin (Robson, M. C. Wound Rep. Reg., vol. 5 (1997), 12-17). Collagenase, a protein critical in wound remodeling, is also produced in response to PDGF (Steed, D. L. Surg. Clin. North Am., vol. 77 (1997), 575-586).

PDGF is also involved in pathological conditions, such as tumorogenesis, arteriosclerosis, rheumatoid arthritis, pulmonary fibrosis, myelofibrosis or abnormal wound repair (Bornfeldt et al., Ann. NY Acad. Sci., vol. 766 (1995), 416-430; Heldin, C. H., FEBS Lett., vol. 410 (1997), 17-21) and acts as a mitogen for bone cells which stimulate the proliferation of osteoblastic cells (Horner et al., Bone, vol. 19 (1996), 353-362.

### SUMMARY OF THE INVENTION

The invention is based, in part, on the discovery that PDGF can be produced in the milk of a transgenic animal. There are three known isoforms of PDGF, each a homo- or heterodimeric combination of two peptide chains designated A and B. The three dimeric isoforms of PDGF are PDGF-AA, PDGF-AB and PDGF-BB. PDGF is active as a dimer, either homo- or heterodimer. It was discovered that PDGF produced in the milk of transgenic animals is in active, e.g., dimeric, form.

Accordingly in one aspect, the invention features a method of producing transgenic PDGF or a preparation of transgenic PDGF. The method includes:

providing a transgenic non-human animal, e.g., a transgenic non-human mammal, which includes a nucleic acid sequence including a nucleic acid sequence encoding PDGF operably linked to a mammary gland specific promoter; and

allowing the PDGF to be expressed in the milk of the transgenic animal, to thereby produce transgenic PDGF.

In a preferred embodiment, all or some of the PDGF in the milk of the transgenic animal is in active form, e.g., all or some of the PDGF in the milk of the transgenic animal is in the form of a dimer.

In a preferred embodiment, the method further includes recovering the transgenically produced PDGF or a preparation of transgenically produced PDGF, from the milk of the animal.

In another preferred embodiment, the method further includes:

inserting a nucleic acid which includes a nucleic acid sequence encoding PDGF, and optionally a mammary gland specific promoter, into a cell and allowing the cell to give rise to a transgenic animal. For example, the nucleic acid sequence can be inserted into an oocyte, e.g., a fertilized oocyte, or a somatic cell, e.g., a fibroblast.

In a preferred embodiment, the transgenic mammals can be selected from: ruminants; ungulates; domesticated mammals; and dairy animals. Preferred mammals include: goats, sheep, mice, cows, pigs, horses, oxen, and rabbits.

In a preferred embodiment, the transgenically produced PDGF preparation, preferably as it is made in the transgenic animal, is glycosylated. In a preferred embodiment, the transgenically produced PDGF differs in its glycosylation pattern from PDGF as it is found or as it is isolated from naturally occurring nontransgenic source, or as it is isolated from recombinantly produced PDGF in cell culture.

In a preferred embodiment, the nucleic acid sequence encoding PDGF encodes a PDGF-A chain. In a preferred embodiment, the PDGF is expressed in the milk as a dimer, e.g., the PDGF is expressed in the milk as a PDGF-AA homodimer. In a preferred embodiment, when the nucleic acid sequence encoding PDGF encodes the PDGF-A chain, at least 30%, 40%, 50%, 55%, 60%, 65%, 70%, 75%, 80%, 85%, 90%, 95%, 98%, 99% or all of the PDGF in the milk is as a dimer, e.g., a PDGF-AA homodimer.

In a preferred embodiment, the nucleic acid sequence encoding PDGF encodes a PDGF-B chain. In a preferred embodiment, the PDGF is expressed in the milk as a dimer, e.g., the PDGF is expressed in the milk as a PDGF-BB homodimer. In a preferred embodiment, when the nucleic acid sequence encoding PDGF encodes the PDGF-B chain at least 30%, 40%, 50%, 55%, 60%, 65%, 70%, 75%, 80%, 85%, 90%, 95%, 98%, 99% or all of the PDGF in the milk is as a dimer, e.g., a PDGF-BB homodimer.

In a preferred embodiment, the transgenic animal includes a nucleic acid sequence encoding PDGF-A chain and a nucleic acid sequence encoding PDGF-B chain. The nucleic acid sequence can include both the PDGF-A encoding sequence and the PDGF-B encoding sequence. The nucleic acid sequence can further include: one mammary gland specific promoter which directs expression of both the PDGF-A encoding sequence and the PDGF-B encoding sequence; two mammary gland specific promoters, one which directs the expression of the PDGF-A encoding sequence and one which directs expression of the PDGF-B encoding sequence. When the nucleic acid sequence includes two mammary gland specific promoters, the mammary gland specific promoters can be the same mammary gland specific promoter or different mammary gland specific promoters.

In another preferred embodiment, the transgenic animal can include two separate nucleic acid sequences, one including a PDGF-A encoding sequence under the control of a mammary gland specific promoter and the other including a PDGF-B encoding sequence under the control of a mammary gland specific promoter. The mammary gland specific promoter linked to the PDGF-A encoding sequence can be the same mammary gland specific promoter as linked to the PDGF-B encoding sequence (e.g., both nucleic acid sequences can include a β-casein promoter) or the sequence encoding PDGF-A can be operably linked to a different mammary gland specific promoter than the sequence encoding PDGF-B (e.g., the PDGF-A encoding sequence is linked to a β-casein promoter and the PDGF-B encoding sequence is linked to a mammary gland specific promoter other than the β-casein promoter).

In a preferred embodiment, where the transgenic animal includes a nucleic acid sequence encoding a PDGF-A chain and a nucleic acid sequence encoding a PDGF-B chain, the milk of the transgenic animal includes: PDGF-AB heterodimers; PDGF-AA homodimers; PDGF-BB homodimers; combinations thereof. In a preferred embodiment, at least 30%, 40%, 50%, 55%, 60%, 65%, 70%, 75%, 80%, 85%, 90%, 95%, 98%, 99% or all of the PDGF in the milk is as a dimer, e.g., a homodimer and/or heterodimer. In a preferred embodiment, at least 30%, 40%, 50%, 55%, 60%, 65%, 70%, 75%, 80%, 85%, 90%, 95%, 98%, 99or all of the PDGF dimers in the milk are PDGF-AB heterodimers. In another preferred embodiment, at least 30%, 40%, 50%, 55%, 60%, 65%, 70%, 75%, 80%, 85%, 90%, 95%, 98%, 99% or all of the PDGF dimers in the milk are homodimers, e.g., PDGF-AA and/or PDGF-BB. In yet another embodiment, less than 95%, 90%, 85%, 80%, 75%, 70%, 65%, 60%, 55%, 50%, 40%, 30%, 20%, 10%, 5%, 1% of the PDGF dimers in the milk are PDGF-AB heterodimers. In another preferred embodiment, embodiment, less than 95%, 90%, 85%, 80%, 75%, 70%, 65%, 60%, 55%, 50%, 40%, 30%, 20%, 10%, 5%, 1% of the PDGF dimers in the milk are homodimers, e.g., PDGF-AA and/or PDGF-BB.

In a preferred embodiment, the milk of a transgenic animal having a PDGF-A encoding sequence and a PDGF-B encoding sequence has: a ratio of total homodimers, e.g., PDGF-AA and/or PDGF-BB, to heterodimers, e.g., PDGF-AB, which is greater than 1, 2, 3, 4, 5. In a preferred embodiment, the milk of the transgenic animal has ratio of homodimers, e.g., PDGF-AA and/or PDGF-BB, to heterodimers, e.g., PDGF-AB, wherein: there is a greater number homodimers, e.g., PDGF-AA and/or PDGF-BB, than heterodimers, e.g., PDGF-AB; there is a greater number of heterodimers, e.g., PDGF-AB, than homodimers, e.g., PDGF-AA and/or PDGF-BB. In another preferred embodiment, the milk of the transgenic animal has: a greater number of PDGF-BB homodimers than PDGF-AA homodimers and/or PDGF-AB heterodimers; a greater number of PDGF-AA homodimers than PDGF-BB homodimers and/or PDGF-AB heterodimers.

In preferred embodiments, the mammary gland specific promoter can be: a casein promoter, beta lactoglobulin promoter, whey acid protein promoter, or lactalbumin promoter.

In preferred embodiments, the transgenically produced PDGF preparation differs in activity from PDGF as it is found or as it is isolated from recombinantly produced PDGF in cell culture, e.g., in yeast cell culture.

In preferred embodiments, the PDGF is mammalian or primate PDGF, preferably human PDGF.

In preferred embodiments, the preparation includes at least 1, 5, 10, 100, or 500 milligrams per milliliter of PDGF.

In another aspect, the invention features, a method for providing a transgenic preparation which includes PDGF in the milk of a transgenic mammal including:

obtaining milk from a transgenic mammal having introduced into its germline a nucleic acid sequence encoding PDGF operatively linked to a promoter sequence that results in the expression of the sequence encoding PDGF in mammary gland epithelial cells, thereby secreting the PDGF in the milk of the mammal to provide the preparation.

In a preferred embodiment, all or some of the PDGF in the milk of the transgenic animal is in active form, e.g., all or some of the PDGF in the milk of the transgenic animal is in the form of a dimer.

In a preferred embodiment, the method further includes recovering the transgenically produced PDGF or a preparation of transgenically produced PDGF, from the milk of the animal.

In a preferred embodiment, the transgenic mammals can be selected from: ruminants; ungulates; domesticated mammals; and dairy animals. Preferred mammals include: goats, sheep, mice, cows, pigs, horses, oxen, and rabbits.

In a preferred embodiment, the transgenically produced PDGF preparation, preferably as it is made in the transgenic animal, is glycosylated. In a preferred embodiment, the transgenically produced PDGF differs in its glycosylation pattern from PDGF as it is found or as it is isolated from naturally occurring nontransgenic source, or as it is isolated from recombinantly produced PDGF in cell culture.

In a preferred embodiment, the PDGF encoding sequence is a PDGF-A chain encoding sequence. In a preferred embodiment, the PDGF is expressed in the milk as a dimer, e.g., the PDGF is expressed in the milk as a PDGF-AA homodimer. In a preferred embodiment, when the PDGF coding sequence encodes the PDGF-A chain, at least 30%, 40%, 50%, 55%, 60%, 65%, 70%, 75%, 80%, 85%, 90%, 95%, 98%, 99% or all of the PDGF in the milk is as a dimer, e.g., a PDGF-AA homodimer.

In a preferred embodiment, the PDGF encoding sequence is a PDGF-B chain encoding sequence. In a preferred embodiment, the PDGF is expressed in the milk as a dimer, e.g., the PDGF is expressed in the milk as a PDGF-BB homodimer. In a preferred embodiment, when the nucleic acid sequence encoding PDGF encodes the PDGF-B chain at least 30%, 40%, 50%, 55%, 60%, 65%, 70%, 75%, 80%, 85%, 90%, 95%, 98%, 99% or all of the PDGF in the milk is as a dimer, e.g., a PDGF-BB homodimer.

In a preferred embodiment, the transgenic animal includes a nucleic acid sequence encoding a PDGF-A chain and a nucleic acid sequence encoding a PDGF-B chain. The nucleic acid sequence can include both the PDGF-A encoding sequence and the PDGF-B encoding sequence. The nucleic acid sequence can further include: one mammary gland specific promoter which directs expression of both the PDGF-A encoding sequence and the PDGF-B encoding sequence; two mammary gland specific promoters, one which directs the expression of the PDGF-A encoding sequence and one which directs expression of the PDGF-B encoding sequence. When the nucleic acid sequence includes two mammary gland specific promoters, the mammary gland specific promoters can be the same mammary gland specific promoter or different mammary gland specific promoters.

In another preferred embodiment, the transgenic animal can include two separate nucleic acid sequences, one including a PDGF-A encoding sequence under the control of a mammary gland specific promoter and another which includes a PDGF-B encoding sequence under the control of a mammary gland specific promoter. The mammary gland specific promoter linked to the PDGF-A encoding sequence can be the same mammary gland specific promoter as linked to the PDGF-B encoding sequence (e.g., both nucleic acid sequences include a β-casein promoter) or the sequence encoding PDGF-A can be operably linked to a different mammary gland specific promoter than the sequence encoding PDGF-B (e.g., the PDGF-A encoding sequence is linked to a β-casein promoter and the PDGF-B encoding sequence is linked to a mammary gland specific promoter other than the β-casein promoter).

In a preferred embodiment, where the transgenic animal includes a nucleic acid sequence encoding a PDGF-A chain and a nucleic acid sequence encoding a PDGF-B chain, the milk of the transgenic animal includes: PDGF-AB heterodimers; PDGF-AA homodimers; PDGF-BB homodimers; combinations thereof. In a preferred embodiment, at least 30%, 40%, 50%, 55%, 60%, 65%, 70%, 75%, 80%, 85%, 90%, 95%, 98%, 99% or all of the PDGF in the milk is as a dimer, e.g., a homodimer and/or heterodimer.

In another preferred embodiment, at least 30%, 40%, 50%, 55%, 60%, 65%, 70%, 75%, 80%, 85%, 90%, 95%, 98%, 99% or all of the PDGF dimers in the milk are homodimers, e.g., PDGF-AA and/or PDGF-BB. In yet another embodiment, less than 95%, 90%, 85%, 80%, 75%, 70%, 65%, 60%, 55%, 50%, 40%, 30%, 20%, 10%, 5%, 1% of the PDGF dimers in the milk are PDGF-AB heterodimers. In another preferred embodiment, embodiment, less than 95%, 90%, 85%, 80%, 75%, 70%, 65%, 60%, 55%, 50%, 40%, 30%, 30%, 20%, 10%, 5%, 1% of the PDGF dimers in the milk are homodimers, e.g., PDGF-AA and/or PDGF-BB.

In a preferred embodiment, the milk of a transgenic animal having a PDGF-A encoding sequence and a PDGF-B encoding sequence has: a ratio of total homodimers, e.g., PDGF-AA and/or PDGF-BB, to heterodimers, e.g., PDGF-AB, which is greater than 1, 2, 3, 4, or 5. In a preferred embodiment, the milk of the transgenic animal has ratio of homodimers, e.g., PDGF-AA and/or PDGF-BB, to heterodimers, e.g., PDGF-AB, wherein: there is a greater number homodimers, e.g., PDGF-AA and/or PDGF-BB, than heterodimers, e.g., PDGF-AB; there is a greater number of heterodimers, e.g., PDGF-AB, than homodimers, e.g., PDGF-AA and/or PDGF-BB. In another preferred embodiment, the milk of the transgenic animal has: a greater number of PDGF-BB homodimers than PDGF-AA homodimers and/or PDGF-AB heterodimers; a greater number of PDGF-AA homodimers than PDGF-BB homodimers and/or PDGF-AB heterodimers.

In preferred embodiments, the mammary gland specific promoter can be: a casein promoter, beta lactoglobulin promoter, whey acid protein promoter, or lactalbumin promoter.

In preferred embodiments, the transgenically produced PDGF preparation differs in activity from PDGF as it is found or as it is isolated from recombinantly produced PDGF in cell culture, e.g., in yeast cell culture.

In preferred embodiments, the PDGF is mammalian or primate PDGF, preferably human, PDGF.

In preferred embodiments, the preparation includes at least 1, 5, 10, 100, or 500 milligrams per milliliter of PDGF.

In another aspect, the invention features a transgenically produced PDGF preparation, e.g., a PDGF preparation described herein.

In a preferred embodiment, the PDGF is obtained from the milk of a transgenic mammal and all or some of the PDGF obtained from the milk of the transgenic animal is in active form, e.g., all or some of the PDGF in the milk of the transgenic animal is in the form of a dimer, without further dimerization processing.

In a preferred embodiment, the transgenically produced PDGF preparation, preferably as it is made in the transgenic animal, is glycosylated. In a preferred embodiment, the transgenically produced PDGF differs in its glycosylation pattern from PDGF as it is found or as it is isolated from naturally occurring nontransgenic source, or as it is isolated from recombinantly produced PDGF in cell culture.

In a preferred embodiment, the PDGF is expressed in the milk as a dimer, e.g., the PDGF is expressed in the milk as a PDGF-AA homodimer or a PDGF-BB homodimer. In a preferred embodiment, at least 30%, 40%, 50%, 55%, 60%, 65%, 70%, 75%, 80%, 85%, 90%, 95%, 98%, 99% or all of the PDGF in the milk is as a dimer, e.g., a PDGF-AA homodimer or a PDGF-BB homodimer. In another preferred embodiment, the milk of the transgenic mammal includes: PDGF-AB heterodimers; PDGF-AA homodimers; PDGF-BB homodimers; combinations thereof. In a preferred embodiment, at least 30%, 40%,50%, 55%, 60%, 65%, 70%, 75%, 80%, 85%, 90%, 95%, 98%, 99% or all of the PDGF in the milk is as a dimer, e.g., a homodimer and/or heterodimer. In a preferred embodiment, 30%, 40%, 50%, 55%, 60%, 65%, 70%, 75%, 80%, 85%, 90%, 95%, 98%, 99% or all of the PDGF dimers in the milk are PDGF-AB heterodimers. In another preferred embodiment, at least 30%, 40%, 50%, 55%, 60%, 65%, 70%, 75%, 80%, 85%, 90%, 95%, 98%, 99% or all of the PDGF dimers in the milk are homodimers, e.g., PDGF-AA and/or PDGF-BB. In yet another embodiment, less than 95%, 90%, 85%, 80%, 75%, 70%, 65%, 60%, 55%, 50%, 40%, 30%, 20%, 10%, 5%, 1% of the PDGF dimers in the milk are PDGF-AB heterodimers. In another preferred embodiment, embodiment, less than 95%, 90%, 85%, 80%, 75%, 70%, 65%, 60%, 55%, 50%, 40%, 30%, 20%, 10%, 5%, 1% of the PDGF dimers in the milk are homodimers, e.g., PDGF-AA and/or PDGF-BB.

In a preferred embodiment, the milk of a transgenic animal having a PDGF-A encoding sequence and a PDGF-B encoding sequence has: a ratio of total homodimers, e.g., PDGF-AA and/or PDGF-BB, to heterodimers, e.g., PDGF-AB, which is greater than 1, 2, 3, 4, 5. In a preferred embodiment, the milk of the transgenic animal has ratio of homodimers, e.g., PDGF-AA and/or PDGF-BB, to heterodimers, e.g., PDGF-AB, wherein: there is a greater number homodimers, e.g., PDGF-AA and/or PDGF-BB, than heterodimers, e.g., PDGF-AB; there is a greater number of heterodimers, e.g., PDGF-AB, than homodimers, e.g., PDGF-AA and/or PDGF-BB. In another preferred embodiment, the milk of the transgenic animal has: a greater number of PDGF-BB homodimers than PDGF-AA homodimers and/or PDGF-AB heterodimers; a greater number of PDGF-AA homodimers than PDGF-BB homodimers and/or PDGF-AB heterodimers.

In preferred embodiments, the transgenically produced PDGF preparation differs in activity from PDGF as it is found or as it is isolated from recombinantly produced PDGF in cell culture, e.g., in yeast cell culture.

In preferred embodiments, the PDGF is mammalian or primate PDGF, preferably human, PDGF.

In preferred embodiments, the preparation includes at least 1, 5, 10, 100, or 500 milligrams per milliliter of PDGF.

In another aspect, the invention features an isolated nucleic acid molecule including a nucleic acid sequence encoding PDGF operatively linked to a tissue specific promoter, e.g., a mammary gland specific promoter sequence that results in the secretion of the protein in the milk of a transgenic mammal.

In preferred embodiments, the promoter is a mammary gland specific promoter, e.g., a milk serum protein or casein promoter. The mammary gland specific promoter can is a casein promoter, beta lactoglobulin promoter, whey acid protein promoter, or lactalbumin promoter.

In preferred embodiments, the nucleic acid sequence encodes mammalian or primate PDGF, preferably human PDGF.

In a preferred embodiment, the PDGF encoding sequence is: a PDGF-A chain encoding sequence; a PDGF-B chain encoding sequence.

In another preferred embodiment, the nucleic acid sequence includes PDGF-A chain encoding sequence and a PDGF-B chain encoding sequence. The nucleic acid sequence can further include: one mammary gland specific promoter which directs expression of both the PDGF-A encoding sequence and the PDGF-B encoding sequence; two mammary gland specific promoters, one which directs the expression of the PDGF-A encoding sequence and one which directs expression of the PDGF-B encoding sequence. When the nucleic acid sequence includes two mammary gland specific promoters, the mammary gland specific promoters can be the same mammary gland specific promoter or different mammary gland specific promoters.

In another aspect, the invention features, a transgenic animal, e.g., a transgenic mammal, which expresses transgenic PDGF, preferably human PDGF, and from which a transgenic preparation of PDGF can be obtained.

Preferably, the transgenic animal is a transgenic mammal. Suitable mammals include: ruminants; ungulates; domesticated mammals; and dairy animals. Particularly preferred animals include: goats, sheep, mice, cows, pigs, horses, oxen, and rabbits. Where the transgenic protein is secreted into the milk of a transgenic animal, the animal should be able to produce at least 1, and more preferably at least 10, or 100, liters of milk per year.

In a preferred embodiment, the transgenic animal secretes PDGF into its milk.

In a preferred embodiment, the transgenic animal produces glycosylated PDGF. In a preferred embodiment, the transgenic animal produces PDGF which differs in its glycosylation pattern from PDGF as it is found or as it is isolated from naturally occurring nontransgenic source, or as it is isolated from recombinantly produced PDGF in cell culture.

In a preferred embodiment, the transgenic animal has a nucleic acid sequence which includes a PDGF-A chain encoding sequence. In a preferred embodiment, the transgenic animal expresses in its milk as a dimer, e.g., the PDGF is expressed in the milk as a PDGF-AA homodimer. In a preferred embodiment, when the animal has a PDGF coding sequence which encodes the PDGF-A chain, at least 30%, 40%, 50%, 55%, 60%, 65%, 70%, 75%, 80%, 85%, 90%, 95%, 98%, 99% or all of the PDGF in its milk is as a dimer, e.g., a PDGF-AA homodimer.

In a preferred embodiment, the transgenic animal has a nucleic acid sequence which includes a PDGF-B chain encoding sequence. In a preferred embodiment, the transgenic animal expresses PDGF in its milk as a dimer, e.g., the PDGF is expressed in the milk as a PDGF-BB homodimer. In a preferred embodiment, when the animal has a PDGF coding sequence which encodes the PDGF-B chain at least 30%, 40%, 50%, 55%, 60%, 65%, 70%, 75%, 80%, 85%, 90%, 95%, 98%, 99% or all of the PDGF in its milk is as a dimer, e.g., a PDGF-BB homodimer.

In a preferred embodiment, the transgenic animal includes a nucleic acid sequence encoding PDGF-A chain and a nucleic acid sequence encoding PDGF-B chain. The nucleic acid sequence can include both the PDGF-A encoding sequence and the PDGF-B encoding sequence. The nucleic acid sequence can further include: one mammary gland specific promoter which directs expression of both the PDGF-A encoding sequence and the PDGF-B encoding sequence; two mammary gland specific promoters, one which directs the expression of the PDGF-A encoding sequence and one which directs expression of the PDGF-B encoding sequence. When the nucleic acid sequence includes two mammary gland specific promoters, the mammary gland specific promoters can be the same mammary gland specific promoter or different mammary gland specific promoters.

In another preferred embodiment, the transgenic animal can include two separate nucleic acid sequences, one including a PDGF-A encoding sequence under the control of a mammary gland specific promoter and another which includes a PDGF-B encoding sequence under the control of a mammary gland specific promoter. The mammary gland specific promoter linked to the PDGF-A encoding sequence can be the same mammary gland specific promoter as linked to the PDGF-B encoding sequence (e.g., both nucleic acid sequences include a β-casein promoter) or the sequence encoding PDGF-A can be operably linked to a different mammary gland specific promoter than the sequence encoding PDGF-B (e.g., the PDGF-A encoding sequence is linked to a β-casein promoter and the PDGF-B encoding sequence is linked to a mammary gland specific promoter other than the β-casein promoter).

In a preferred embodiment, when the transgenic animal includes a nucleic acid sequence encoding PDGF-A chain and a nucleic acid sequence encoding PDGF-B chain, the milk of the transgenic animal includes: PDGF-AB heterodimers; PDGF-AA homodimers; PDGF-BB homodimers; combinations thereof. In a preferred embodiment, at least 30%, 40%, 50%, 55%, 60%, 65%, 70%, 75%, 80%, 85%, 90%, 95%, 98%, 99% or all of the PDGF in its milk is as a dimer, e.g., a homodimer and/or heterodimer. In a preferred embodiment, 30%, 40%, 50%, 55%, 60%, 65%, 70%, 75%, 80%, 85%, 90%, 95%, 98%, 99% or all of the PDGF dimers in its milk are PDGF-AB heterodimers. In another preferred embodiment, at least 30%, 40%, 50%, 55%, 60%, 65%, 70%, 75%, 80%, 85%, 90%, 95%, 98%, 99% or all of the PDGF dimers in the milk are homodimers, e.g., PDGF-AA and/or PDGF-BB. In yet another embodiment, less than 95%, 90%, 85%, 80%, 75%, 70%, 65%, 60%, 55%, 50%, 40%, 30%, 20%, 10%, 5%, 1% of the PDGF dimers in the milk are PDGF-AB heterodimers. In another preferred embodiment, embodiment, less than 95%, 90%, 85%, 80%, 75%, 70%, 65%, 60%, 55%, 50%, 40%, 30%, 20%, 10%, 5%, 1% of the PDGF dimers in the milk are homodimers, e.g., PDGF-AA and/or PDGF-BB.

In a preferred embodiment, the milk of a transgenic animal having a PDGF-A encoding sequence and a PDGF-B encoding sequence has: a ratio of total homodimers, e.g., PDGF-AA and/or PDGF-BB, to heterodimers, e.g., PDGF-AB, which is greater than 1, 2, 3, 4, 5. In a preferred embodiment, the milk of the transgenic animal has ratio of homodimers, e.g., PDGF-AA and/or PDGF-BB, to heterodimers, e.g., PDGF-AB, wherein: there is a greater number homodimers, e.g., PDGF-AA and/or PDGF-BB, than heterodimers, e.g., PDGF-AB; there is a greater number of heterodimers, e.g., PDGF-AB, than homodimers, e.g., PDGF-AA and/or PDGF-BB. In another preferred embodiment, the milk of the transgenic animal has: a greater number of PDGF-BB homodimers than PDGF-AA homodimers and/or PDGF-AB heterodimers; a greater number of PDGF-AA homodimers than PDGF-BB homodimers and/or PDGF-AB heterodimers.

In preferred embodiments, the transgenic animal expresses PDGF in its milk at levels of at least 1, 5, 10, 100, or 500 milligrams per milliliter of PDGF.

In another aspect, the invention features, a pharmaceutical composition including a therapeutically effective amount of transgenic PDGF, or a transgenic preparation of PDGF, and a pharmaceutically acceptable carrier.

The transgenic PDGF or PDGF preparation can be made, e.g., by any method or animal described herein.

The transgenic PDGF or PDGF preparation can be, e.g., any described herein.

In another aspect, the invention features, a method of providing transgenically produced PDGF, e.g., any PDGF described herein, to a subject in need of PDGF. The method includes: administering transgenically produced PDGF or a transgenic preparation of PDGF to the subject.

In preferred embodiments the subject is: a person, e.g., a patient, in need of PDGF.

For example, the invention features a method for stimulating or enhancing wound healing in a subject. The wound can be in soft tissue or hard tissue, e.g., bone. In a preferred embodiment, transgenically produced PDGF stimulates or enhances would healing by one or more of the biological activities of PDGF. Biological activities of PDGF include: 1) modulation, e.g., induction, of extracellular matrix synthesis; 2) modulation, e.g., increasing, of hyaluronic acid and fibronectin production; 3) modulation, e.g., increasing, of collagenase production; 4) mitogenic effect for connective tissue and/or mesenchymal derived cells; 5) modulation of, e.g., increasing or decreasing, migration of blood cells, e.g., neutrophils and/or monocytes; 6) modulation of, e.g., increasing or decreasing, migration of fibroblasts; 7) modulation, e.g., induction, of the clotting cascade, e.g., it induces expression of tissue factor which initiates clotting cascade; 7) modulation of, e.g., increasing, actin reorganization; and 8) it mitogenic effect for bone cells, e.g., it modulates, e.g., increases, proliferation of osteoblastic cells.

The structure of transgenic PDGF can be modified for such purposes as enhancing therapeutic or prophylactic efficacy, or stability (e.g., ex vivo shelf life and resistance to proteolytic degradation in vivo), or to optimize the health of the animal. Such modified PDGF, when designed to retain at least one activity of the natural PDGF, are considered functional equivalents of the PDGF described in more detail herein. Such modified peptide can be produced, for instance, by amino acid substitution, deletion, or addition.

A preparation, as used herein, refers to two or more molecules of PDGF. The preparation can be produced by one or more than one transgenic animal. It can include molecules of differing glycosylation or it can be homogenous in this regard.

A purified preparation, substantially pure preparation of a polypeptide, or an isolated polypeptide as used herein, means, in the case of a transgenically produced polypeptide, a polypeptide that has been separated from at least one other protein, lipid, or nucleic acid with which it occurs in the transgenic animal or in a fluid, e.g., milk, or other substance produced by the transgenic animal. The polypeptide is preferably separated from substances, e.g., antibodies or gel matrix, e.g., polyacrylamide, which are used to purify it. The polypeptide is preferably constitutes at least 10, 20, 50 70, 80 or 95% dry weight of the purified preparation. Preferably, the preparation contains: sufficient polypeptide to allow protein sequencing; at least 1, 10, or 100 μg of the polypeptide; at least 1, 10, or 100 mg of the polypeptide.

As used herein, the term transgene means a nucleic acid sequence (encoding, e.g., one or more PDGF polypeptides), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression and secretion of the selected nucleic acid encoding PDGF, e.g., in a mammary gland, all operably linked to the selected PDGF nucleic acid, and may include an enhancer sequence. The PDGF sequence can be operatively linked to a tissue specific promoter, e.g., mammary gland specific promoter sequence that results in the secretion of the protein in the milk of a transgenic mammal.

As used herein, the term “transgenic cell” refers to a cell containing a transgene.

As used herein, a “transgenic animal” is a non-human animal in which one or more, and preferably essentially all, of the cells of the animal contain a heterologous nucleic acid introduced by way of human intervention, such as by transgenic techniques known in the art. The transgene can be introduced into the cell, directly or indirectly by introduction into a precursor of the cell, by way of deliberate genetic manipulation, such as by microinjection or by infection with a recombinant virus.

Mammals are defined herein as all animals, excluding humans, that have mammary glands and produce milk.

The term “pharmaceutically acceptable composition” refers to compositions which comprise a therapeutically effective amount of transgenic PDGF, formulated together with one or more pharmaceutically acceptable carrier(s).

As used herein, the language “subject” is intended to include human and non-human animals.

Other features and advantages of the invention will be apparent from the following detailed description, and from the claims.

### BRIEF DESCRIPTION OF THE DRAWINGS

FIG. 1: depicts the nucleic acid sequence of the PDGF-AB insert of expression vector pBC734. This sequence includes the nucleic acid sequence encoding human PDGF A chain, an IRES and a nucleic acid sequence encoding human PDGF B chain. This 2 kb insert was ligated into the mammary gland expression vector pBC450 (nucleic acid sequence provided), to create the expression cassette pBC734. The nucleic acid sequence of the PDGF-B insert of expression vector pBC701 is also provided. This insert was ligated into the mammary gland expression vector pBC450 (nucleic acid sequence provided), to create the expression cassette pBC701.

### DETAILED DESCRIPTION OF THE INVENTION

### Transgenic Mammals

Methods for generating non-human transgenic mammals are known in the art. Such methods can involve introducing DNA constructs into the germ line of a mammal to make a transgenic mammal. For example, one or several copies of the construct may be incorporated into the genome of a mammalian embryo by standard transgenic techniques. In addition, non-human transgenic mammals can be produced using a somatic cell as a donor cell. The genome of the somatic cell can then be inserted into an oocyte and the oocyte can be fused and activated to form a reconstructed embryo. For example, methods of producing transgenic animals using a somatic cell are described in PCT Publication WO 97/07669; Baguisi et al. Nature Biotech., vol. 17 (1999), 456-461; Campbell et al., Nature, vol. 380 (1996), 64-66; Cibelli et al., Science, vol. 280 (1998); Kato et al., Science, vol. 282 (1998), 2095-2098; Schnieke et al., Science, vol. 278. (1997), 2130-2133; Wakayama et al., Nature, vol. 394 (1998), 369-374; Well et al., Biol. Reprod., vol. 57 (1997):385-393.

Although goats are a preferred source of genetically engineered cells, other non-human mammals can be used. Preferred non-human mammals are ruminants, e.g., cows, sheep, or goats. Goats of Swiss origin, e.g., the Alpine, Saanen and Toggenburg breed goats, are useful in the methods described herein. Additional examples of preferred non-human animals include oxen, horses, llamas, and pigs. The mammal used as the source of genetically engineered cells will depend on the transgenic mammal to be obtained by the methods of the invention as, by way of example, a goat genome should be introduced into a goat functionally enucleated oocyte.

Preferably, for cloning, the somatic cells are obtained from a transgenic goat. Methods of producing transgenic goats are known in the art. For example, a transgene can be introduced into the germline of a goat by microinjection as described, for example, in Ebert et al. (1994) Bio/Technology 12:699, hereby incorporated by reference.

Other transgenic non-human animals to be used as a source of genetically engineered somatic cells can be produced by introducing a transgene into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor.

### Transfected Cell Lines

Genetically engineered cell lines can be used to produce a transgenic animal. A genetically engineered construct can be introduced into a cell via conventional transformation or transfection techniques. As used herein, the terms “transfection” and “transformation” include a variety of techniques for introducing a transgenic sequence into a host cell, including calcium phosphate or calcium chloride co-precipitation, DEAE-dextrane-mediated transfection, lipofection, or electroporation. In addition, biological vectors, e.g., viral vectors can be used as described below. Suitable methods for transforming or transfecting host cells can be found in Sambrook et al., Molecular Cloning: A Laboratory Manuel, 2^{nd} ed., Cold Spring Harbor Laboratory, (Cold Spring Harbor Laboratory Press, Cold Spring Harbor, N.Y., 1989), and other suitable laboratory manuals.

Two useful approaches are electroporation and lipofection. Brief examples of each are described below.

The DNA construct can be stably introduced into a donor cell line by electroporation using the following protocol: somatic cells, e.g., fibroblasts, e.g., embryonic fibroblasts, are resuspended in PBS at about 4×10⁶ cells/ml. Fifty micrograms of linearized DNA is added to the 0.5 ml cell suspension, and the suspension is placed in a 0.4 cm electrode gap cuvette (Biorad). Electroporation is performed using a Biorad Gene Pulser electroporator with a 330 volt pulse at 25 mA, 1000 microFarad and infinite resistance. If the DNA construct contains a neomyocin resistance gene for selection, neomyocin resistant clones are selected following incubation with 350 microgram/ml of G418 (GibcoBRL) for 15 days.

The DNA construct can be stably introduced into a donor somatic cell line by lipofection using a protocol such as the following: about 2×10⁵ cells are plated into a 3.5 cmiameter well and transfected with 2 micrograms of linearized DNA using LipfectAMINE™ (GibcoBRL). Forty-eight hours after transfection, the cells are split 1:1000 and 1:5000 and, if the DNA construct contains a neomyosin resistance gene for selection, G418 is added to a final concentration of 0.35 mg/ml. Neomyocin resistant clones are isolated and expanded for cyropreservation as well as nuclear transfer.

### Tissue-Specific Expression of Proteins

It is often desirable to express a heterologous protein, e.g., a PDGF, in a specific tissue or fluid, e.g., the milk, of a transgenic animal. The heterologous protein can be recovered from the tissue or fluid in which it is expressed. For example, it is often desirable to express the heterologous protein in milk. Methods for producing a heterologous protein under the control of a mammary gland specific promoter are described below. In addition, other tissue-specific promoters, as well as, other regulatory elements, e.g., signal sequences and sequence which enhance secretion of non-secreted proteins, are described below.

### Mammary Gland Specific Promoters

Useful transcriptional promoters are those promoters that are preferentially activated in mammary epithelial cells, including promoters that control the genes encoding milk proteins such as caseins, beta lactoglobulin (Clark et al., (1989) Bio/Technology 7: 487-492), whey acid protein (Gordon et al. (1987) Bio/Technology 5: 1183-1187), and lactalbumin (Soulier et al., (1992) FEBS Letts. 297: 13). Casein promoters may be derived from the alpha, beta, gamma or kappa casein genes of any mammalian species; a preferred promoter is derived from the goat beta casein gene (DiTullio, (1992) Bio/Technology 10:74-77). The promoter can also be from lactoferrin or butyrophin. Mammary gland specific protein promoter or the promoters that are specifically activated in mammary tissue can be derived from cDNA or genomic sequences. Preferably, they are genomic in origin.

DNA sequence information is available for the mammary gland specific genes listed above, in at least one, and often in several organisms. See, e.g., Richards et al., J. Biol. Chem. 256, 526-532 (1981) (α-lactalbumin rat); Campbell et al., Nucleic Acids Res. 12, 8685-8697 (1984) (rat WAP); Jones et al., J. Biol. Chem. 260, 7042-7050 (1985) (rat β-casein); Yu-Lee & Rosen, J. Biol. Chem. 258, 10794-10804 (1983) (rat γ-casein); Hall, Biochem. J. 242, 735-742 (1987) (α-lactalbumin human); Stewart, Nucleic Acids Res. 12, 389 (1984) (bovine αs1 and κ casein cDNAs); Gorodetsky et al., Gene 66, 87-96 (1988) (bovine β casein); Alexander et al., Eur. J. Biochem. 178, 395-401 (1988) (bovine κ casein); Brignon et al., FEBS Lett. 188, 48-55 (1977) (bovine αS2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine β lactoglobulin); Vilotte et al., Biochimie 69, 609-620 (1987) (bovine α-lactalbumin). The structure and function of the various milk protein genes are reviewed by Mercier & Vilotte, J. Dairy Sci. 76, 3079-3098 (1993) (incorporated by reference in its entirety for all purposes). If additional flanking sequences are useful in optimizing expression of the heterologous protein, such sequences can be cloned using the existing sequences as probes. For example, the nucleic acid can also include an enhancer sequence. Mammary-gland specific regulatory sequences from different organisms can be obtained by screening libraries from such organisms using known cognate nucleotide sequences, or antibodies to cognate proteins as probes.

### Signal Sequences

Useful signal sequences are milk-specific signal sequences or other signal sequences which result in the secretion of eukaryotic or prokaryotic proteins. Preferably, the signal sequence is selected from milk-specific signal sequences, i.e., it is from a gene which encodes a product secreted into milk. Most preferably, the milk-specific signal sequence is related to the mammary gland specific promoter used in the construct, which are described below. The size of the signal sequence is not critical. All that is required is that the sequence be of a sufficient size to effect secretion of the desired recombinant protein, e.g., in the mammary tissue. For example, signal sequences from genes coding for caseins, e.g., alpha, beta, gamma or kappa caseins, beta lactoglobulin, whey acid protein, and lactalbumin can be used. A preferred signal sequence is the goat β-casein signal sequence.

Signal sequences from other secreted proteins, e.g., proteins secreted by kidney cells, pancreatic cells or liver cells, can also be used. Preferably, the signal sequence results in the secretion of proteins into, for example, urine or blood. Examples of other genes from which the signal sequence can be derived include: serum albumin (human, bovine murine, caprine, ovine), tissue plasminogen activator (human, bovine murine, caprine, ovine), alpha-1-antitrypsin (human, bovine murine, caprine, ovine), growth hormone (human, bovine murine, caprine, ovine, murine, rat), and immunoglobulins. Any of these or other signal sequences may be inserted in the nucleic acid of the present invention.

### Amino-Terminal Regions of Secreted Proteins

A non-secreted protein can also be modified in such a manner that it is secreted such as by inclusion in the protein to be secreted of all or part of the coding sequence of a protein which is normally secreted. Preferably the entire sequence of the protein which is normally secreted is not included in the sequence of the protein but rather only a sufficient portion of the amino terminal end of the protein which is normally secreted to result in secretion of the protein. For example, a protein which is not normally secreted is fused (usually at its amino terminal end) to an amino terminal portion of a protein which is normally secreted.

In one aspect, the protein which is normally secreted is a protein which is normally secreted in milk. Such proteins include proteins secreted by mammary epithelial cells, milk proteins such as caseins, beta lactoglobulin, whey acid protein, lactoferrin, butyrophillin and lactalbumin. Casein proteins include alpha, beta, gamma or kappa casein genes of any mammalian species. A preferred protein is beta casein, e.g., goat beta casein. The sequences which encode the secreted protein can be derived from either cDNA or genomic sequences. Preferably, they are genomic in origin, and include one or more introns.

### Other Tissue-Specific Promoters

Other tissue-specific promoters which provide expression in a particular tissue can be used. Tissue specific promoters are promoters which are expressed more strongly in a particular tissue than in others. Tissue specific promoters are often expressed essentially exclusively in the specific tissue.

Tissue-specific promoters which can be used include: a neural-specific promoter, e.g., nestin, Wnt-1, Pax-1, Engrailed-1, Engrailed-2, Sonic hedgehog; a liver-specific promoter, e.g., albumin, alpha-1 antitrypsin; a muscle-specific promoter, e.g., myogenin, actin, MyoD, myosin; an oocyte specific promoter, e.g., ZP1, ZP2, ZP3; a testes-specific promoter, e.g., protamin, fertilin, synaptonemal complex protein-1; a blood-specific promoter, e.g., globulin, GATA-1, porphobilinogen deaminase; a lung-specific promoter, e.g., surfactant protein C; a skin- or wool-specific promoter, e.g., keratin, elastin; endothelium-specific promoters, e.g., Tie-1, Tie-2; and a bone-specific promoter, e.g., BMP.

In addition, general promoters can be used for expression in several tissues. Examples of general promoters include β-actin, ROSA-21, PGK, FOS, c-myc, Jun-A, and Jun-B.

### Other Regulatory Sequences

The nucleic acid may also include a DNA sequence 3′ of the PDGF coding sequence which is referred to herein as the 3′ regulatory sequence. The 3′ regulatory sequence can include a 3′ untranslated region (UTR) and/or a 3′ flanking sequence. The 3′ UTR and the 3′ flanking sequence can be from the same gene or a different gene, or from the same species or from different species. In a preferred embodiment, the 3′ regulatory sequence is derived from a mammalian milk gene.

### Insulator Sequences

The DNA constructs used to make a transgenic animal can include at least one insulator sequence. The terms “insulator”, “insulator sequence” and “insulator element” are used interchangeably herein. An insulator element is a control element which insulates the transcription of genes placed within its range of action but which does not perturb gene expression, either negatively or positively. Preferably, an insulator sequence is inserted on either side of the DNA sequence to be transcribed. For example, the insulator can be positioned about 200 bp to about 1 kb, 5′ from the promoter, and at least about 1 kb to 5 kb from the promoter, at the 3′ end of the gene of interest. The distance of the insulator sequence from the promoter and the 3′ end of the gene of interest can be determined by those skilled in the art, depending on the relative sizes of the gene of interest, the promoter and the enhancer used in the construct. In addition, more than one insulator sequence can be positioned 5′ from the promoter or at the 3′ end of the transgene. For example, two or more insulator sequences can be positioned 5′ from the promoter. The insulator or insulators at the 3′ end of the transgene can be positioned at the 3′ end of the gene of interest, or at the 3 ′ end of a 3′ regulatory sequence, e.g., a 3′ untranslated region (UTR) or a 3′ flanking sequence.

A preferred insulator is a DNA segment which encompasses the 5′ end of the chicken β-globin locus and corresponds to the chicken 5′ constitutive hypersensitive site as described in PCT Publication 94/23046, the contents of which is incorporated herein by reference.

### DNA Constructs

A cassette which encodes a heterologous protein can be assembled as a construct which includes a promoter for a specific tissue, e.g., for mammary epithelial cells, e.g., a casein promoter, e.g., a goat beta casein promoter, a milk-specific signal sequence, e.g., a casein signal sequence, e.g., a β-casein signal sequence, and a DNA encoding the heterologous protein.

The construct can also include a 3′ untranslated region downstream of the DNA sequence coding for the non-secreted protein. Such regions can stabilize the RNA transcript of the expression system and thus increases the yield of desired protein from the expression system. Among the 3′ untranslated regions useful in the constructs for use in the invention are sequences that provide a poly A signal. Such sequences may be derived, e.g., from the SV40 small t antigen, the casein 3′ untranslated region or other 3′ untranslated sequences well known in the art. In one aspect, the 3′ untranslated region is derived from a milk specific protein. The length of the 3′ untranslated region is not critical but the stabilizing effect of its poly A transcript appears important in stabilizing the RNA of the expression sequence.

Optionally, the construct can include a 5′ untranslated region between the promoter and the DNA sequence encoding the signal sequence. Such untranslated regions can be from the same control region from which promoter is taken or can be from a different gene, e.g., they may be derived from other synthetic, semi-synthetic or natural sources. Again their specific length is not critical, however, they appear to be useful in improving the level of expression.

The construct can also include about 10%, 20%, 30%, or more of the N-terminal coding region of a gene preferentially expressed in mammary epithelial cells. For example, the N-terminal coding region can correspond to the promoter used, e.g., a goat β-casein N-terminal coding region.

The construct can be prepared using methods known in the art. The construct can be prepared as part of a larger plasmid. Such preparation allows the cloning and selection of the correct constructions in an efficient manner. The construct can be located between convenient restriction sites on the plasmid so that they can be easily isolated from the remaining plasmid sequences for incorporation into the desired mammal.

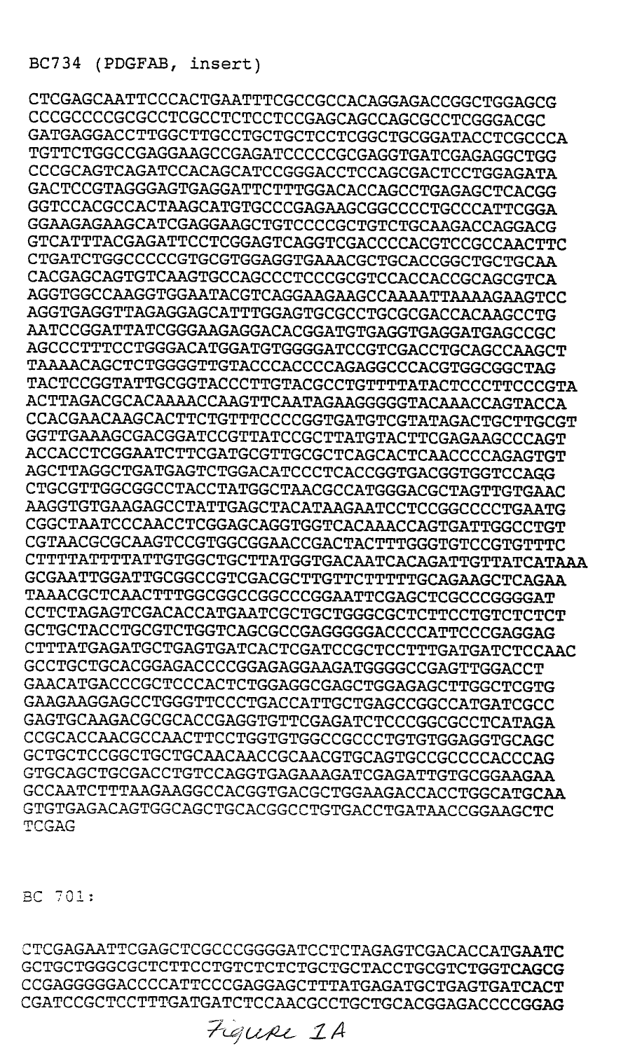

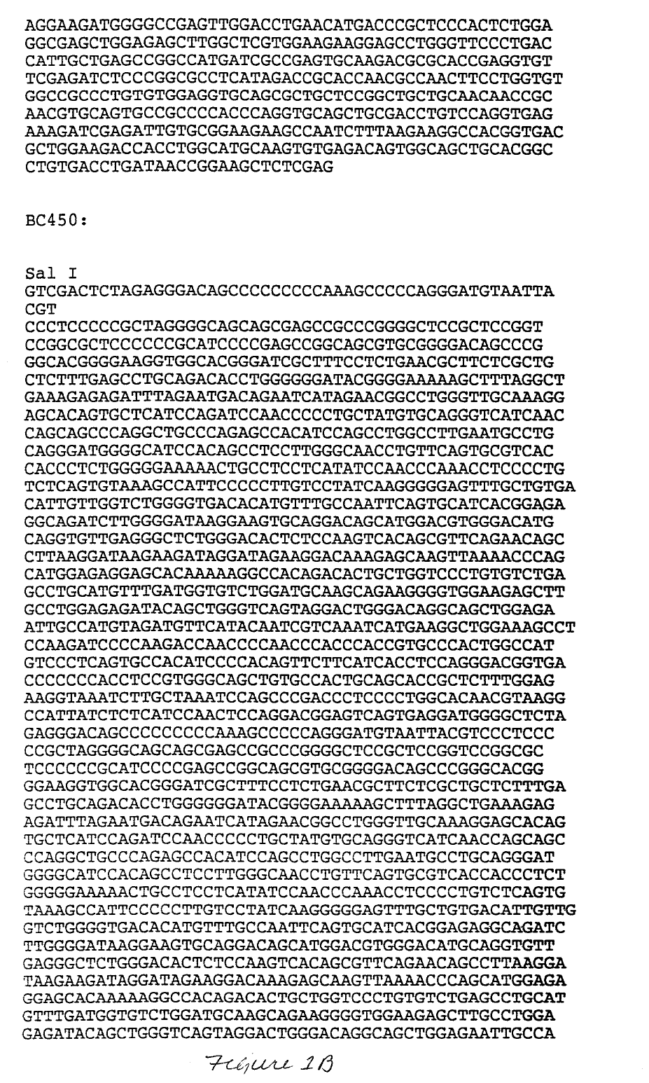

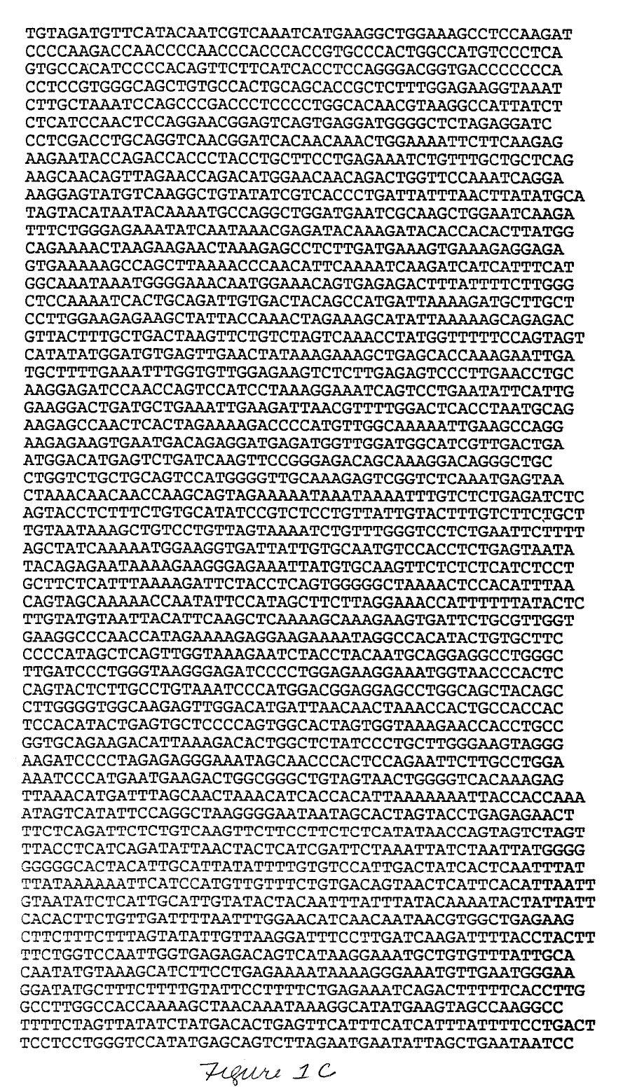

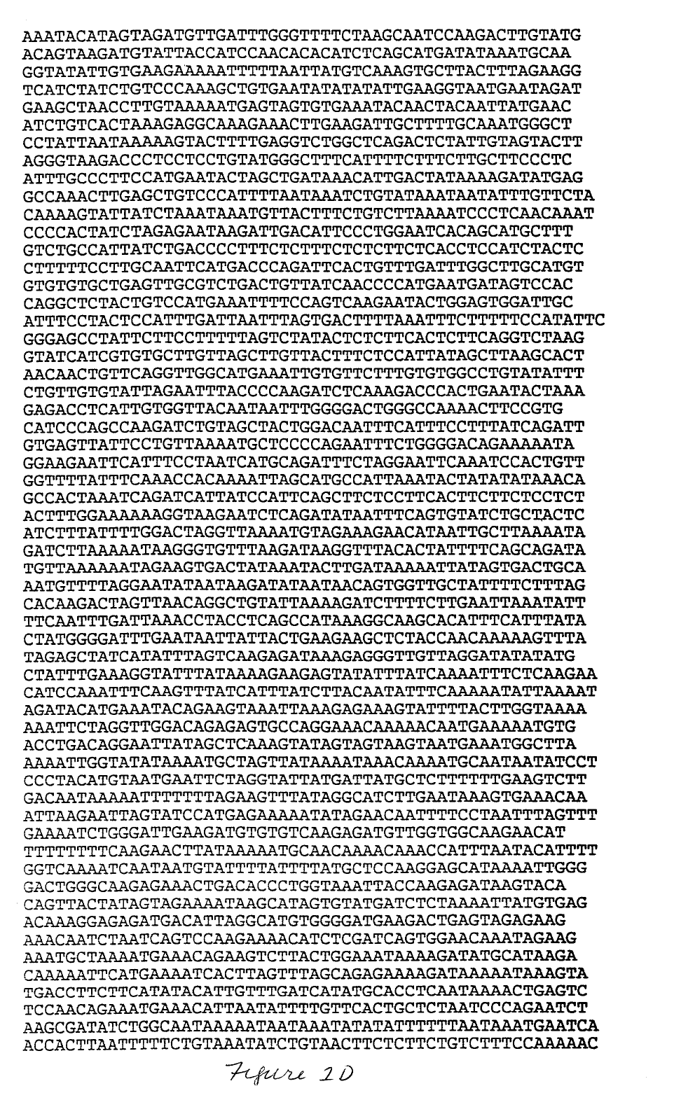

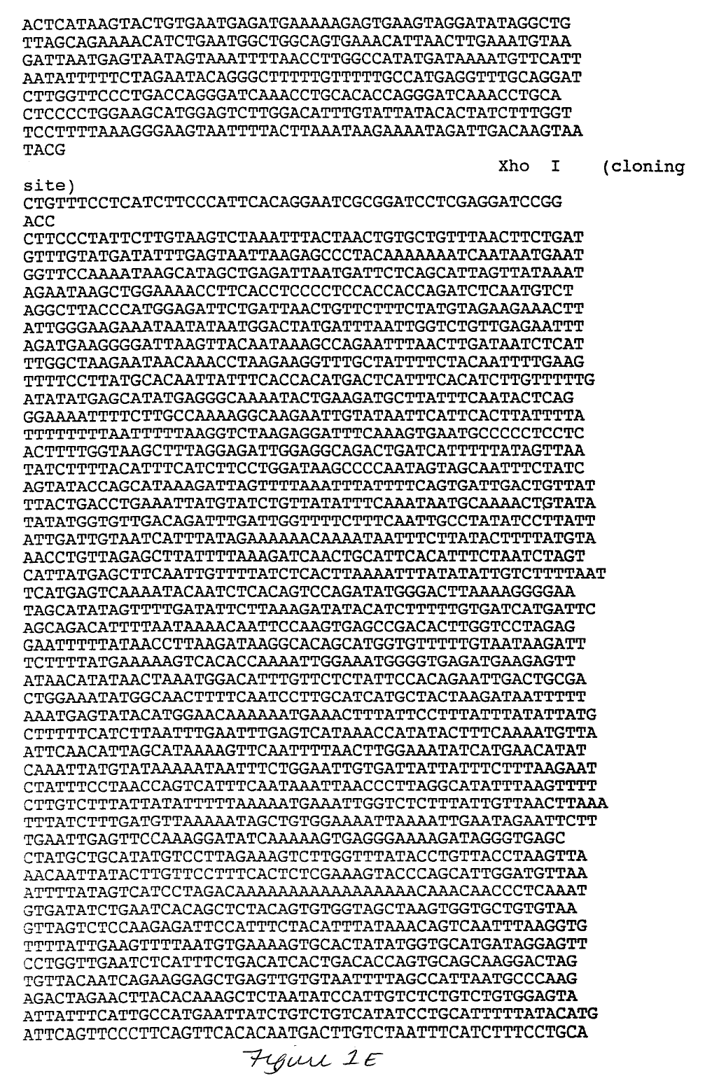

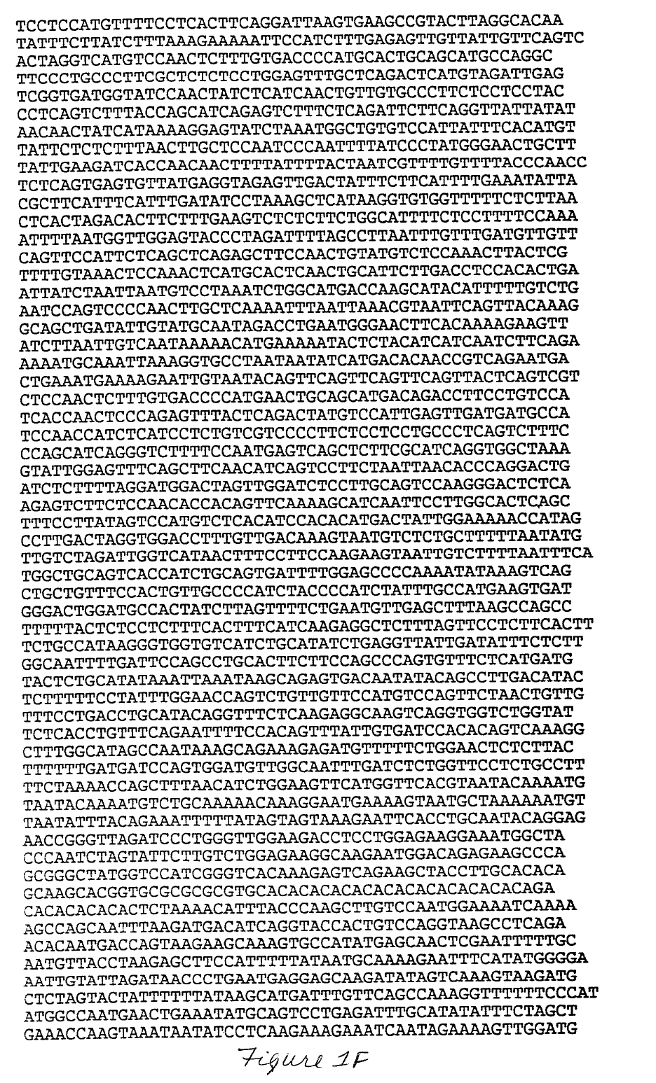

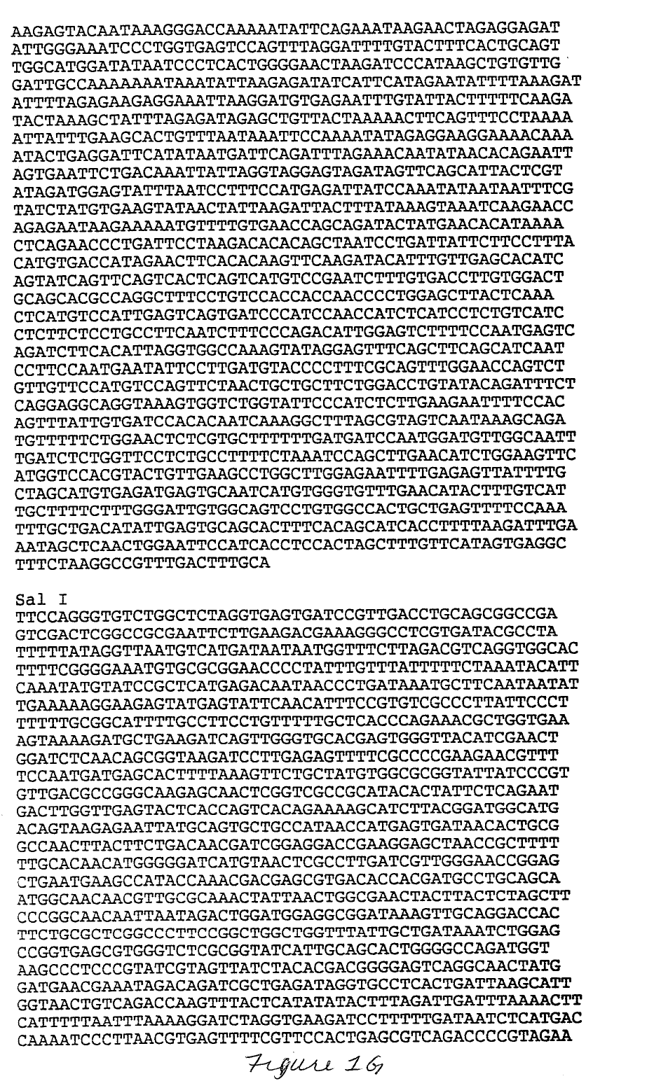

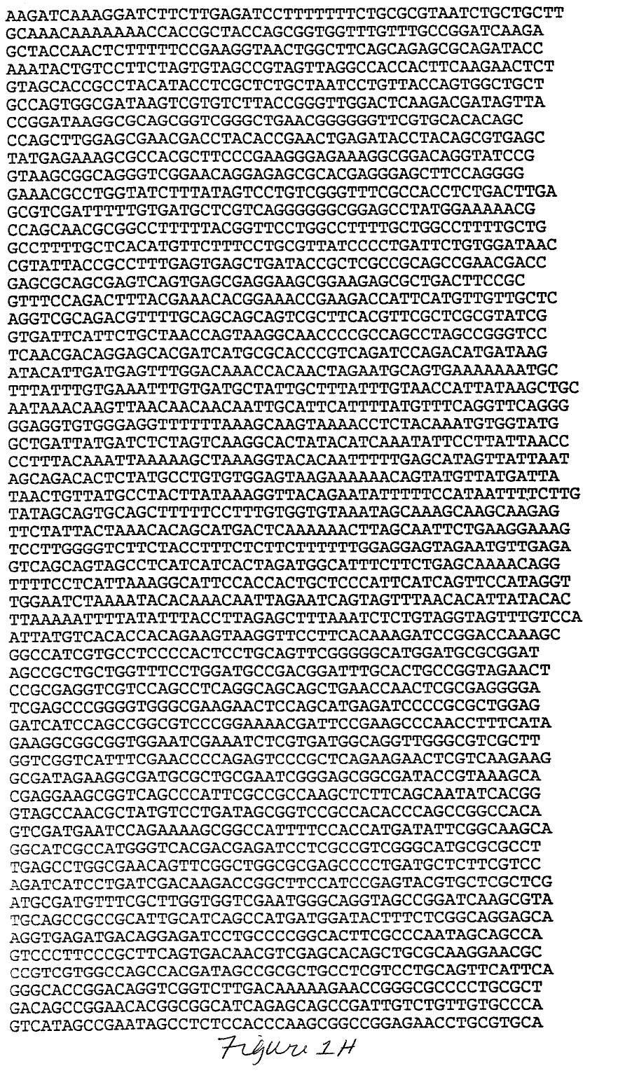

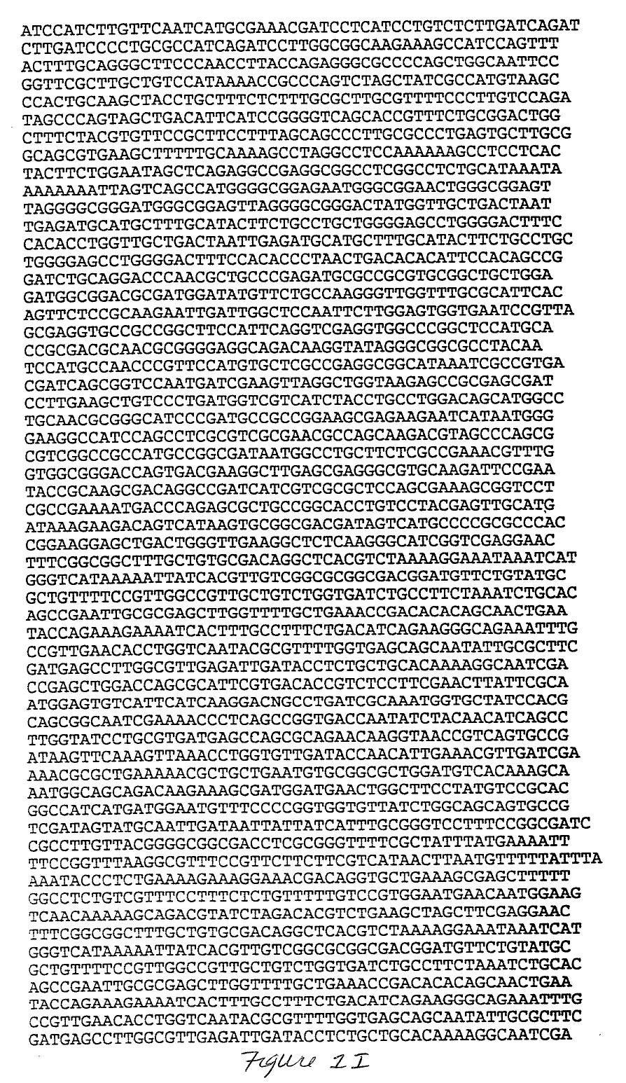

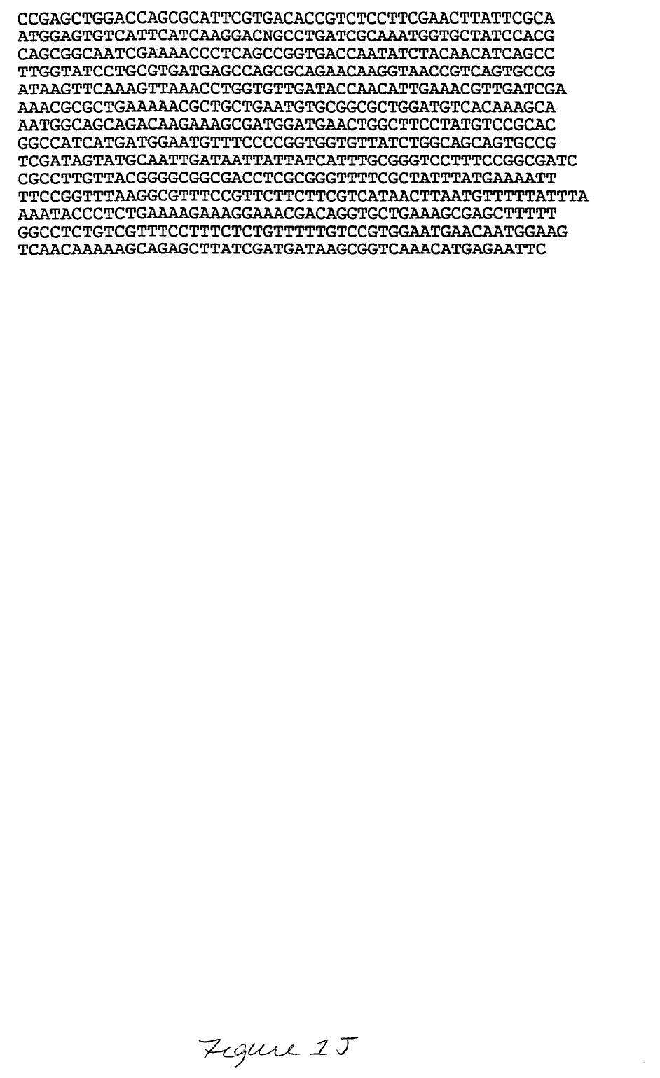

A nucleic acid sequence encoding PDGF can be introduced into a mammary gland expression plasmid, e.g., a plasmid which includes a mammary gland specific promoter. Examples of mammary gland expression plasmids are BC701 and BC734 described in the examples below. Organization of the BC701 and BC734 mammary gland expression cassettes are shown in FIG. 1. In both cassettes the transgene is flanked by NotI restriction sites (on both sides).

The expression plasmid including the sequence encoding PDGF may also include one or more origins of replication and/or selection markers.

### Platelet Derived Growth Factor (PDGF) and Fragments and Analogs Thereof

“PDGF”, as used herein, refers to a growth factor protein, or a fragment or analog thereof having at least one biological activity of PDGF. A polypeptide has PDGF biological activity if it has one or more of the following activities: 1) modulation, e.g., induction, of extracellular matrix synthesis; 2) modulation, e.g., increasing, of hyaluronic acid and fibronectin production; 3) modulation, e.g., increasing, of collagenase production; 4) mitogenic effect for connective tissue and/or mesenchymal derived cells; 5) modulation of, e.g., increasing or decreasing, migration of blood cells, e.g., neutrophils and/or monocytes; 6) modulation of, e.g., increasing or decreasing, migration of fibroblasts; 7) modulation, e.g., induction, of the clotting cascade, e.g., it induces expression of tissue factor which initiates clotting cascade; 8) modulation of, e.g., increasing, actin reorganization; 9) it interacts, e.g., binds, to a PDGF receptor, e.g., a PDGF α and/or β receptor; and 10) it has a mitogenic effect on bone cells, e.g., it modulates, e.g., increases, proliferation of osteoblastic cells. Several assays are available for analyzing if a PDGF has any of the biological activity listed above, e.g. cell proliferation or thymidine incorporation bioassays (Shipley et al., Cancer Research, vol. 44, 710-716). For example, binding of PDGF to its receptor can be demonstrated by numerous methods known in the art. Such methods can include competition assays using iodinated (¹²⁵I) PDGF to determine the ability of a fragment or analog of PDGF to bind its receptor (Hunter, W. M. and Greenwood, F. C., Nature vol. 194 (1962), 495-496).

There are three isoforms of PDGF, PDGF-AA, PDGF-BB and PDGF-AB, which are homo- or heterodimeric combinations of two distinct peptide chains designated A and B (for a review see Meyer-Ingold and Eichner, Cell Biology International, vol. 19 (1995), 389-398). The nucleic acid encoding the A chain and/or the B chain can be a cDNA or genomic sequence encoding the PDGF chain. In other embodiments, a genomic DNA sequence encoding the PDGF A chain and/or B chain can include at least one but not all of the introns naturally present in the genomic PDGF gene.

The PDGF-A chain, as used herein, refers to full length PDGF A-chain or variants, e.g., naturally occurring variants, thereof. For example, various transcripts have been detected in PDGF-AA producing cells. These transcripts are alternative spliced variants of a single seven exon gene of PDGF-A which gives rise to a short (S) and long (L) processed protein of 110 amino acids (A_{S}) and 125 amino acids (A_{L}). The shorter transcript lacks exon 6, which contains 69 base pairs. Characteristics of the PDGF-Aₛ chain are described, for example, in (Matoskova et al., Molecular and Cellular Biology, vol. 9 (1989), 3148-3150). The sequence encoded by exon 6 apparently regulates secretion of PDGF from the producing cell. Exon 6 containing variants are retained in the producing cell while the exon 7 encoded sequence containing short splice variant (Aₛ) is effectively secreted (Feyzi et al., J Biol Chem, vol. 272 (1997), 5518-5524). PGDF-A, as used herein, can refer to PDGF-A_{S} or PDGF-A_{L}. The nucleic acid sequences encoding PDGF-A_{S} and PDGF-A_{L} are known and described, for example, in Rorsman et al., Mol. Cell Biol., vol. 8(2) (1988), 571-577.

The PDGF-B chain, as used herein, refers to the 109 amino acid sequence described, for example, in Östman et al., Journal of Cell Biology, vol. 118 (1992), 509-519, as well as variants, e.g., naturally-occurring variants, thereof. The nucleotide sequence encoding PDGF-B is known and described, for example, in Rao et al., Prot. Nat'l Acad. Sci., vol. 83(8) (1996) 2392-2396.

The nucleic acid sequences described herein can encode human PDGF or PDGF of other mammals (such as cow, monkey, pig, goat, rabbit, etc.). The DNA sequence coding for PDGF can be a cDNA or a genomic DNA sequence. Genomic DNA sequences are generally better expressed in transgenic animals (Hurwitz et al., Transgenic Res., vol. 3 (1994), 365, and Whitelaw et al., Transgenic Res. vol. 1 (1991), 3). Surprisingly, the present invention has achieved high expression of PDGF using a cDNA sequence.

The sequence encoding PDGF can code for the A and/or B isoform of PDGF. Depending on the sequence of the PDGF isoform inserted into the nucleic acid, it is possible to obtain PDGF-AA, -BB or a mixture of all three isoforms (-AA, -BB and -AB). For example, when the nucleic acid sequence encodes a PDGF-A chain, e.g., the nucleic acid sequence is monocistronic for expression of PDGF-A chain, the PDGF can be expressed in the milk as a PDGF-AA homodimer. Preferably, when the nucleic acid sequence encoding PDGF encodes the PDGF-A chain, at least 30%, 40%, 50%, 55%, 60%, 65%, 70%, 75%, 80%, 85%, 90%, 95%, 98%, 99% or all of the PDGF in the milk is as a PDGF-AA homodimer. Alternatively, when the nucleic acid sequence encodes a PDGF-B chain, e.g., the nucleic acid sequence is monocistronic for expression of PDGF-B chain, the PDGF is expressed in the milk as a PDGF-BB homodimer. Preferably, when the nucleic acid sequence encoding PDGF encodes the PDGF-B chain at least 30%, 40%, 50%, 55%, 60%, 65%, 70%, 75%, 80%, 85%, 90%, 95%, 98%, 99% or all of the PDGF in the milk is as a PDGF-BB homodimer.

A transgenic animal can also include a nucleic acid sequence encoding PDGF-A chain and a nucleic acid sequence encoding PDGF-B chain. This animal can be used to produce both PDGF homo and heterodimers. The nucleic acid sequence can include both the PDGF-A encoding sequence and the PDGF-B encoding sequence, e.g., the nucleic acid sequence can be polycistronic, e.g., dicistronic, for expression of PDGF. Polycistronic expression constructs for PDGF have been described, for example, in WO 94/29462 and WO 94/05786, the contents of which are incorporated herein by reference. Such expression constructs can be used to create a transgenic animal which includes a nucleic acid encoding a PDGF-A chain and a PDGF-B chain such that expression of these polypeptides is directed into the mammary gland of the animal. Thus, the nucleic acid sequence can further include one mammary gland specific promoter which directs expression of both the PDGF-A encoding sequence and the PDGF-B encoding sequence (e.g., the nucleic acid sequence can include one mammary gland specific promoter and an IRES) or two mammary gland specific promoters, one which directs the expression of the PDGF-A encoding sequence and one which directs expression of the PDGF-B encoding sequence. When the nucleic acid sequence includes two mammary gland specific promoters, the mammary gland specific promoters can be the same mammary gland specific promoter or different mammary gland specific promoters.

Alternatively, the transgenic animal can include two separate nucleic acid sequences, one including a PDGF-A encoding sequence under the control of a mammary gland specific promoter and the other including a PDGF-B encoding sequence under the control of a mammary gland specific promoter, e.g., the transgenic animal can co-express a nucleic acid sequence which is monocistronic for expression of PDGF-A chain and a nucleic acid sequence which is monocistronic for expression of PDGF-B chain. The mammary gland specific promoter linked to the PDGF-A encoding sequence can be the same mammary gland specific promoter as linked to the PDGF-B encoding sequence (e.g., both nucleic acid sequences can include a β-casein promoter) or the sequence encoding PDGF-A can be operably linked to a different mammary gland specific promoter than the sequence encoding PDGF-B (e.g., the PDGF-A encoding sequence is linked to a β-casein promoter and the PDGF-B encoding sequence is linked to a mammary gland specific promoter other than the β-casein promoter).

Depending on the intended use for the PDGF, it may be desirable to produce a particular PDGF isoform, e.g., either PDGF-AA, PDGF-BB, PDGF-AB or combinations thereof. Each of the PDGF isoforms may have an increased effect on a particular cells type and/or an enhanced or different PDGF activity as compared to the other isoforms. For example, the responsiveness of a cell to the different isoforms is regulated by the expression of known PDGF-receptors. The isoforms of PDGF, PDGF-AA, AB and BB, are differentially expressed in various cell types (Pierce and Mustoe, 1995). The effects of PDGF are mediated through two distinct receptors. These receptors as referred to herein as the α PDGF receptor and the β PDGF receptor. For further discussion of these receptors see, e.g., Gronwald et al., Proceedings of the National Academy of Sciences of the United States of America, vol. 85 (1988), 3435-3439; and Bonner, J. C., Annals of the New York Academy of Sciences, vol. 737 (1994), 324-338). The α receptor binds to all three PDGF isoforms with high affinity, whereas the β receptor binds to the PDGF-BB homodimer with high affinity, to the PDGF-AB heterodimer with lower activity and does not bind to the PDGF-AA homodimer. Hart et al., Science, vol. 240 (1988), 1529-1531.

Both PDGF receptors are highly homologous tyrosine kinases with quite similar structural properties. Dimerization is important in PDGF receptor activation, which allows phosphorylation in trans between the two receptors in the complex. The binding of PDGF isoforms to PDGF receptors has been studied and several amino acid residues have been identified as playing a role in this interaction. For example, complementary to the receptor binding sites, the residues arginine 27 and isoleucine 30 of the PDGF-B chain seem to be important for receptor binding and cell activation of PDGF-BB (Clements et al., EMBO J. vol. 10 (1991), 4113). In addition, autophosphorylation sites on the receptor have been found to provide docking sites for signal transduction molecules.

On cells having the same amount of α-and β-receptors, PDGF-AB has been found to have stronger mitogenic and chemotactic effects than the homodimeric isoforms (Heldin et al., Biochim Biophys Acta, vol. 1378 (1998), F79-113). Most cells, however, have more β-receptors than α-receptors (Steed, D. L., Clin Plast Surg, vol. 25 (1998), 397-405). Since a homodimerization of β-receptors can only be induced by the PDGF-BB isoform, in some embodiments, it may be preferable to produce only the PDGF-BB isoform. In contrast to the β-receptor, α-receptors can bind A- and B-chains of PDGF. The binding regions for PDGF-AA and PDGF-BB on the α-receptor are not, however, structurally coincident (Heldin et al., 1998).

Both receptors share some functional properties. For example, they can both induce mitogenic responses or actin reorganization. In other aspects, the receptors do not share functional properties. For example, the PDGF β-receptor is able to mediate the stimulation of chemotaxis while the α-receptor inhibits the migration of certain cell types. See, e.g., Heldin, C. H., 1997. Thus, the transgenic PDGF isoform to be produced can be decided based on the desired use for the PDGF preparation.

Situations were one isoform may be preferred over another are discussed below. PDGF-BB has been shown to mediate a chemotactic response via β-receptors in human fibroblasts whereas activation of α-receptors by PDGF-BB has been shown to inhibit chemotaxis (Vassbotn et al., J Biol Chem, vol. 267 (1992), 15635-15641). The PDGF-AA isoform is the major form present at the sites of injury during the acute phase of the wound repair response (Soma et al., FASEB J, vol. 6 (1992), 2996-3001). Treatment of chronic wounds with exogenous recombinant PDGF-BB resulted in the appearance of PDGF-AA within capillaries by 2 weeks and was associated with a healing phenotype (Pierce et al., J Clin Invest, vol. 96 (1995), 1336-1350). PDGF-AA splice variants can have unique biological activities and differ in their time of appearance during the repair process (Pierce at al., 1995). In early wound healing, PDGF-AA_{L} has been found to be present in maximal quantities while in the maturing granulation tissue of healing wounds PDGF-AA_{S} is prevalent. The PDGF isoforms share many effects in wound healing but nevertheless a more positive effect of PDGF-BB on rat wound healing was shown in comparison to the usage of corresponding doses of PDGF-AA. In addition, the heterodimeric form of PDGF (PDGF-AB), accelerates dose-dependently granulation tissue formation in experimental wounds in rat (Lepisto et al., Eur. Surg. Res., vol. 26 (1994), 267-272). Thus, a particular isoform of PDGF or combinations thereof can be produced depending on the intended use of the PDGF.

The PDGF produced by a transgenic animal, as described herein, can be a fragment or analog of PDGF which retains at least one biological activity of PDGF. PDGF fragments and analogs can be obtained by recombinant expression of nucleic acid sequences which are related to the natural PDGF sequence. Nucleic acid sequences encoding a fragment or analog of PDGF can be prepared, for example, by modifying a known PDGF nucleotide sequence. Such modifications can include additions, substitutions and/or deletions of any number of nucleotides. Other analogs of PDGF can include a polypeptide which differs from PDGF isolated from tissue in one or more of the following: its pattern of glycosylation, phosphorylation, or other posttranslational modifications. In one embodiment, the transgenically produced PDGF differs in its glycosylation pattern from PDGF as it is found or as it is isolated from a naturally occurring nontransgenic source, or as it is isolated from recombinantly produced PDGF in cell culture. The glycosylation pattern of PDGF can play an important role on the activity of PDGF. For example, it has been shown that hyperglycosylated PDGF as compared to non-glycosylated PDGF had a 2 to 4 fold higher activity. See WO 91/16335. Examples of natural homologs for a sequence encoding PDGF include the v-sis gene isolated from Simian Sarcoma Virus. The v-sis gene encodes a protein which has extensive sequence homology to the B chain of PDGF and as a homodimer is capable of binding to the human PDGF receptor (EP 177 957). Preferably, fragments and analogs of PDGF-A chain and PDGF-B chain retain the ability to form a dimer, e.g., a homo- or heterodimer.

Those skilled in the art can prepare such modified nucleic acids by methods known in the art and described below.

### Production of Fragments and Analogs of PDGF

One skilled in the art can alter the disclosed structure of PDGF by producing fragments or analogs, and test the newly produced structures for activity. Examples of prior art methods which allow the production and testing of fragments and analogs are discussed below. These, or other methods, can be used to make and screen fragments and analogs of a PDGF polypeptide.

### Generation of PDGF Fragments

Fragments of a protein can be produced in several ways, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Expression of the mutagenized DNA produces polypeptide fragments. Digestion with “end-nibbling” endonucleases can thus generate DNA's which encode an array of fragments. DNA's which encode fragments of a protein can also be generated by random shearing, restriction digestion or a combination of the above-discussed methods.

Fragments can also be chemically synthesized using techniques known in the art such as conventional Merrifield solid phase f-Moc or t-Boc chemistry. For example, peptides of the present invention may be arbitrarily divided into fragments of desired length with no overlap of the fragments, or divided into overlapping fragments of a desired length.

### Generation of PDGF Analogs: Production of Altered DNA and Peptide Sequences by Random Methods

Amino acid sequence variants of a protein can be prepared by random mutagenesis of DNA which encodes a protein or a particular domain or region of a protein. Useful methods include PCR mutagenesis and saturation mutagenesis. A library of random amino acid sequence variants can also be generated by the synthesis of a set of degenerate oligonucleotide sequences. (Methods for screening proteins in a library of variants are elsewhere herein.)

### PCR Mutagenesis

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

### Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

### Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

### Generation of Analogs: Production of Altered DNA and Peptide Sequences by Directed Mutagenesis

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

### Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

### Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765[1978]).

### Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

### Combinatorial Mutagenesis

Combinatorial mutagenesis can also be used to generate mutants. E.g., the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

### Oocytes

Oocytes for use in producing a transgenic animal can be obtained at various times during an animal's reproductive cycle. Oocytes at various stages of the cell cycle can be obtained and then induced in vitro to enter a particular stage of meiosis. For example, oocytes cultured on serum-starved medium become arrested in metaphase. In addition, arrested oocytes can be induced to enter telophase by serum activation.

Oocytes can be matured in vitro before they are used to form a reconstructed embryo This process usually requires collecting immature oocytes from mammalian ovaries, e.g., a caprine ovary, and maturing the oocyte in a medium prior to enucleation until the oocyte reaches the desired meiotic stage, e.g., metaphase or telophase. In addition, oocytes that have been matured in vivo can be used to form a reconstructed embryo.

Oocytes can be collected from a female mammal during superovulation. Briefly, oocytes, e.g., caprine oocytes, can be recovered surgically by flushing the oocytes from the oviduct of the female donor. Methods of inducing superovulation in goats and the collection of caprine oocytes are described herein.

### Transfer of Reconstructed Embryos

A reconstructed embryo can be transferred to a recipient doe and allowed to develop into a cloned or transgenic mammal. For example, the reconstructed embryo can be transferred via the fimbria into the oviductal lumen of each recipient doe. In addition, methods of transferring an embryo to a recipient mammal are known in the art and described, for example, in Ebert et al. (1994) Bio/Technology 12:699.

### Purification of PDGF from Milk

PDGF can be isolated from milk using standard protein purification methods known in the art. For example, the milk can initially be clarified. A typical clarification protocol can include the following steps:

(a) diluting milk 2:1 with 2.0 M Arginine-HCl pH 5.5;

(b) spinning diluted sample in centrifuge for approximately 20 minutes at 4-8° C.;

(c) cooling samples for approximately 5 minutes on ice to allow fat sitting on top to solidify;

(d) removing fat pad by “popping” it off the top with a pipette tip; and

(e) decanting of supernatant into a clean tube.

Further purification of PDGF can be achieved, for example, using standard chromatographic procedures for the purification of PDGF known in the art. An efficient purification protocol is described, for example, in Heldin et al. (Nature, vol. 319 (1986), 511-514). Briefly, PDGF is isolated from cell culture supernatant using Sephacryl S-200, Bio-Gel P-150 and HPLC (RP-8) columns in subsequent chromatography steps. Another example for high yield (over 50%) purification of PDGF from cell culture supernatant is disclosed in Eichner et al. (Eur. J. Biochem., vol. 185 (1989), p. 135-140), wherein PDGF-AA secreted from baby hamster kidney cells was isolated using adsorption to controlled pore glass, ammonium sulfate precipitation, Bio-Gel 100 chromatography and reversed-phase HPLC.

Alternatively or additionally, the clarified sample may be further purified by diluting the sample further 1:7 with PBS (this will lower the conductivity of the sample enabling it to be loaded onto an affinity column) and filtering the sample using a syringe and a Millipore Millex®-HV 0.45 μm filter unit. For obtaining highly purified PDGF, the sample may then be loaded onto an affinity column.

### Uses

Pharmaceutical compositions which include PDGF can obtained from the milk of transgenic non-human animals. Such compositions can be used to treat a subject in need of PDGF. For example, PDGF can be used to stimulate or enhance the wound healing processes, e.g., wounds in soft tissue or hard tissue (such as bone). In particular, patients suffering from impaired would healing like diabetic foot ulcers, decubitus ulcers, and venous stasis ulcers can be treated with PDGF obtained from transgenic animals. In addition, the transgenically produced PDGF may be applied for the treatment of periodontal regeneration (Giannobile et al., J. Periodont. Res., vol. 31 (1996), 301-312), stimulation of bone formation (Vikjaer et al., Eur. J. Oral Sci., vol. 105 (1979), ophthalmic diseases or healing of prosthetic vascular grafts (Ombrellaro et al., J. Amer. Coll. Surg., vol. 184 (1997), 49-57).

In addition, PDGF obtained from transgenic animals may further be used for the preparation of a medicament for stimulating or enhancing wound healing. The PDGF may for example be applied using a wound dressing, a cream, an ointment or a spray. A wound dressing may have the form of fibers, sheets, granules or flakes. The transgenically produced PDGF can be incorporated into wound management aids prepared from polysaccharides. Polysaccharides such as D-glucans, cellulose, dextran, (1-3)-β-D-glucans, chitin, chitinosan, alginic acid, hyaluronic acid as well as the derivatized forms thereof, such as sulphated or complex polysaccharides, are known for their ability to interact with receptors on a variety of cells and thereby stimulate wound repair and healing processes (Lloyd et al., Carbohydrate Polymers, vol. 37 (1998), 315-322). For use as a wound dressing, transgenically produced PDGF can be incorporated into polysaccharides which are prepared in form of beads, gels, films, sheets or fibers. The PDGF may also be part of a bioresorbable material, such as membranes, beads, sponges, or depot-formulations. PDGF obtainable from transgenic animals can further be used as the bioactive molecule in an Alkermes depot-formulation which is composed of biodegradable microspheres containing the bioactive molecule. The biodegradable microspheres are made from a matrix of poly-(DL-lactide-goglycolide) (PLGA), a common medical polymer. Alkermes is commercially available as ProLease®

The transgenically produced PDGF can also be used for non-medical applications, for example as a supplement for cell culture media or as a component of diagnostic kits.

### EXAMPLES

### Example 1

### Expression Vector Construction

The two expression cassettes BC701 PDGF-B) and BC734 PDGF-A-IRSG-PDGF-B) were constructed using sequences isolated from pSBC-PDGF-A/-G-B. This expression plasmid is described in detail in the U.S. Pat. No. 5,665,567, the contents of which are incorporated herein by reference.

To create BC701, the vector pSBC-PDGF-A/-G-B was first cut partially with restriction enzyme HindIII and was ligated to the self-annealing cohesive linker HINXHO (sequence: AGCTCTCGAG). Integration of this linker destroys the HindIII site and creates and Xho I site in its place. The plasmid pAB21 which had one copy of HINXHO integrated in the HindIII site located at the 3′ end of the PDGF-B gene was identified using restriction enzyme mapping. Plasmid pAB21 was then partially cut with the restriction enzyme Eco RI and was ligated to the self-annealing cohesive linker ECOXHO (sequence: AATTCTCGAG). Integration of this linker into an EcoRI site creates a Xho I site. The plasmid pAB23 which had one copy of ECOXHO integrated in the EcoRI site located just at the 5′ end of the PDGF-B gene was identified using restriction enzyme mapping. Complete digestion of pAB23 with the restriction enzyme XhoI liberates an approximately 750 bp fragment containing the full sequence of the PDGF-B190 gene. PDGF-B190 is a specific gene construct described in detail in EP 658 198. It codes for a translation product (PDGF-BB), which is identical to fully processed mature PDGF-BB. In the construct a stop codon was introduced in position 191 of the PDGF-B precursor protein. As a result, the carboxy-terminal part of the PDGF-B molecule, which is responsible for the retention of incompletely processed forms, is not expressed.

This fragment (PDGF-B190, corresponding to SEQ ID NO:1) was isolated and cloned into the XhoI site of the mammary gland expression vector pBC450, to create PDGF-B expression cassette pBC701 (see FIG. 1A).

The mammary gland expression vector pBC450 includes nucleotide sequences coding for the chicken β-globin insulator sequence (Chung et al., Cell, vol. 74(1993), 505-514) as well as the goat-β-casein promoter (Roberts et al., Gene, vol. 121 (1992), 255). These sequences of pBC450 are provided in SEQ ID NO 2.

To create the PDGF-A-IRESG-PDGF-B expression cassette, the intermediate vector pAB21 was first digested to completion with the restriction enzyme NotI. The ends were filled with Klenow DNA polymerase and the resulting fragment was self-ligated. In the resulting plasmid, pAB2, the restriction site NotI located in the IRES/G sequence had been destroyed. The intermediate vector pAB2 was then cut partially with the restriction enzyme Eco RI and was ligated to the self-annealing cohesive linker ECONOXHO (sequence: AATTGCTCGAGC). Integration of this linker into an EcoRI site creates and Xho I site while destroying the EcoRI site. The plasmid pAB33 which had one copy of ECONOXHO integrated in the EcoRI site located just at the 5′ end of the PDGF-A gene was identified using restriction enzyme mapping. Complete digestion of pAB33 with the restriction enzyme XhoI liberates an approximately 2 kb fragment containing the full sequence of the PDGF-A gene as well as the full sequence of the PDGF-B 190 gene; both genes were separated by the IRESG sequences. This 2 kb fragment was isolated and ligated into the mammary gland expression vector pBC450, to create the expression cassette pBC734 (FIG. 1).

The inserts of both transgenes (pBC701 and pBC734) were fully sequenced and verified prior to microinjection. The full sequence of the pBC734 insert (PDGFB-IRESG-PDGFA) is shown in SEQ ID NO: 3.

### Example 2

### Preparation of Injection Fragments

The BC701 and BC734 PDGF expression cassettes were prepared for microinjection using the “Wizard” method. In each case, plasmid DNA (100 μg) was separated from the vector backbone by digesting to completion with the restriction enzyme NotI. The digests were then electrophoresed in an agarose gel, using 1× TAE (Maniatis et al., 1982) as running buffer. The regions of the gels containing the DNA fragments corresponding to the expression cassettes were visualized under UV light (long wave). The bands containing the DNAs of interest were excised, transferred to a dialysis bag, and the DNAs were isolated by electroelution in 1× TAE.

Following electroelution, the DNA fragments were concentrated and cleaned-up by using the “Wizard DNA clean-up system” (Promega, Cat #A7280), following the protocol provided therewith. The DNA was eluted in 125 microliter of microinjection buffer (10 mM Tris, pH 7.5, 0.2 mM EDTA). Fragment concentrations were evaluated by comparative agarose gel electrophoresis. The DNA stocks were diluted in microinjection buffer just prior to pronuclear injections so that the final concentrations were 1.5 ng/ml.

### Example 3

### Microinjection

CD1 female mice were superovulated and fertilized ova were retrieved from the oviduct. Male pronuclei were then microinjected with DNA diluted in microinjection buffer.

Microinjected embryos were either cultured overnight in CZB media prepared according to Chatot et al. (Journal of Reproduction & Fertility, vol. 86 (1989), 679-688) or transferred immediately into the oviduct of pseudopregnant recipient CD1 female mice. Twenty to thirty 2-cell or forty to fifty one-cell embryos were transferred to each recipient female and allowed to continue to term.

### Example 4

### Identification of Founder Animals

Genomic DNA was isolated from tail tissue by precipitation with isopropanol and analyzed by polymerase chain reaction (PCR) for the presence the chicken beta-globin insulator DNA sequence. For the PCR reactions, approximately 250 ng of genomic DNA were diluted in 5O μl of PCR buffer (20 mM Tris, pH 8.3, 5O mM KCl and 1.5 mM MgCl₂, 100 μM deoxynucleotide triphosphates, and each primer in a concentration of 600 nM) with 2.5 units of Taq polymerase and processed using the following temperature program:

| | | |
|---|---|---|
| 1. cycle | 94° C. | 60 sec |
| 5 cycles | 94° C. | 30 sec |
| | 58° C. | 45 sec |
| | 74° C. | 45 sec |
| 30. cycle | 94° C. | 30 sec |
| | 55° C. | 30 sec |
| | 74° C. | 30 sec |

The following primers were used:

| | | |
|---|---|---|
| GBC 332: | TGTGCTCCTCTCCATGCTGG | (SEQ ID NO:1) |
| GBC 386: | TGGTCTGGGGTGACACATGT | (SEQ ID NO:2) |

A total of 2586 embryos transformed with the BC701 construct were transferred to 76 pseudopregnant recipient mice. A total of 583 founder mice were born (22.5% of transferred embryos) and were analyzed by PCR using primers specific for the insulator sequence. A total of 38 transgenic founders were identified, 23 of which were selected for mating.

### Example 5

### Breeding of Founder Animals

Twenty-three BC701 founders (animals No. 45, 47, 157, 365, 431, 434, 443, 483, 484, 490, 519, 556, 576, 578, 590, 594, 604, 615, 621, 622, 647, 649, 673) were mated. Passage of the transgene to the next generation was observed for 18 lines. Females 443, 490, 519 and 615 did not transmit the transgene to the next generation (probably transgene mosaics). First generation offspring from 18 transgenic lines (45, 47, 157, 365, 431, 434, 483, 484, 556, 576, 578, 590, 594, 604, 621, 622, 649, 673) were mated, and milk was collected from some females. Table 1 summarizes the breeding of each BC701 line.

TABLE 1
Breeding of BC 701 transgenic founders.

| | PCR positive | ID number of |
|---|---|---|
| Founder | offspring/litter | selected F1 |
| (sex) | (females only) | transgenic female |
| 45 (M) | 2/13 | 264,269 |
| 47 (F) | 6/8 | 278,688 |
| 157 (F) | 2/14 | 346,347 |
| 365 (F) | 2/14 | 415,418 |
| 431 (M) | 8/15 | 774,775 |
| 434 (M) | 3/6 | 792,793 |
| 443 (F) | 0/5 | — |
| 483 (F) | 3/9 | 801,804 |
| 484 (F) | 7/8 | 892,894 |
| 490 (F) | 0/2 | — |
| 519 (F) | 0/13 | — |
| 556 (M) | 3/4 | 832,833 |
| 576 (M) | 2/8 | 826,828 |
| 578 (M) | 3/5 | 837,839 |
| 590 (M) | 2/8 | 843,848 |
| 594 (F) | 2/6 | 853,854 |
| 604 (M) | 4/6 | 856,857 |
| 615 (F) | 0/1 | — |
| 621 (M) | 4/6 | 862,864 |
| 622 (M) | 2/10 | 871,877 |
| 648 (M) | 2/5 | 884,888 |
| 649 (F)* | not available | not available |
| 673 (M) | 1/3 | 891 |

All offspring were analyzed with the insulator PCR assay

### Example 6

### Obtaining Milk From Transgenic Mice

Female mice were allowed to deliver their pups naturally, and were generally milked twice between days 6 and 12 postpartum. Mice were separated from their litters for approximately one hour prior to the milking procedure. After the one hour holding period, mice were induced to lactate using an intraperitoneal injection of 5 i.U. Oxytocin in sterile Phosphate Buffered Saline, using a 25 gauge needle. Hormone injections were followed by a waiting period for one to five minutes to allow the Oxytocin to take effect.

A suction and collection system consisting of a 15 ml conical tube sealed with a rubber stopper with two 18 gauge needles inserted in it, the hub end of one needle being inserted into rubber tubing connected to a human breast pump, was used for milking. Mice were placed on a cage top, held only by their tail and otherwise not restricted or confined. The hub end of the other needle was placed over the mice's teats (one at a time) for the purpose of collecting the milk into individual eppendorf tubes placed in the 15 ml conical tube. Eppendorf tubes were changed after each sample collection. Milking was continued until at least 150 μl of milk had been obtained. After collection, mice were returned to their litters.

The method for isolation of PDGF from milk comprises a clarification of the milk. The clarification protocol comprises the steps of:

(a) diluting milk 2:1 with 2.0 M Arginine-HCl pH 5.5;

(b) spinning diluted sample in centrifuge for approximately 20 minutes at 4-8° C.;

(c) cooling samples for approximately 5 minutes on ice to allow fat sitting on top to solidify;

(d) removing fat pad by “popping” it off the top with a pipette tip; and

(e) decanting of supernatant into a clean tube.

The clarified sample is further purified by diluting the sample further 1:7 with PBS (this will lower the conductivity of the sample enabling it to be loaded onto an affinity column) and filtering the sample using a syringe and a Millipore Millex®-HV 0.45 μm filter unit. The sample is then loaded onto an affinity column.

Further purification of PDGF may be achieved using standard chromatographic procedures for the purification of PDGF well known in the art.

### Example 7

### Protein Analysis

Western Blot and biological activity analyses were carried out with the PDGF isolated from the milk of transgenic animals.

In more detail, the Western Blot was probed using the ELISA method with a rabbit polyclonal anti-PDGF-B-antibody (from R&D Systems) as first and goat-anti-rabbit-HRP conjugate as second antibody. Detection was performed using the ECL chemiluminescence system (Pharmacia/Amersham) according to the manufacturer's instructions.

Biological activity analyses were performed using a bioassay, wherein DNA synthesis or thymidine incorporation was assayed in BALBc/3T3 cells according to Weich et al. (Growth Factors, vol. 2 (1990), 313-320) or Klagsbrun & Ching (PNAS, vol. 82 (1985), 805-809).

Milk samples from BC701 transgenic females were analyzed using PDGF-B western-blot and activity assays. It was determined that PDGF-B is expressed at a level of approximately 2-4 mg/ml in the milk of the founder female 647, and to a level of 0.5-1 mg/ml in the milk of the 484 female.

This demonstrates that biologically active recombinant PDGF can be obtained at high levels from the milk of animals transformed with a nucleic acid comprising a DNA sequence encoding a biologically active PDGF operatively linked to a regulatory sequence capable of directing the expression of PDGF in the mammary gland of non-human transgenic mammals.

All patents and references cited herein are incorporated in their entirety by reference. Other embodiments are within the following claims.

What is claimed:

1. A method of producing platelet derived growth factor (PDGF), comprising:
providing a transgenic mammal whose somatic and germ cells comprise a nucleic acid sequence encoding PDGF operably linked to a promoter which directs expression into mammary gland epithelial cells; and
obtaining milk from the transgenic mammal, wherein at least 30% of the PDGF in the milk is as a dimer.

2. The method of claim 1, wherein the nucleic acid sequence encodes a PDGF A chain and at least 30% of the PDGF in the milk is as a PDGF-AA homodimer.

3. The method of claim 1, wherein the nucleic acid sequence encodes a PDGF B chain and at least 30% of the PDGF in the milk is as a PDGF-BB homodimer.

4. The method of claim 1, wherein the nucleic acid sequence comprises a nucleic acid sequence encoding a PDGF A chain and a nucleic acid sequence encoding a PDGF-B chain.

5. The method of claim 4, wherein the nucleic acid sequence encoding the PDGF A chain and the nucleic acid sequence encoding the PDGF B chain are under control of the same promoter.

6. The method of claim 4, wherein the nucleic acid sequence encoding the PDGF A chain is operably linked to a different promoter than the nucleic acid sequence encoding the PDGF B chain.

7. The method of claim 1, wherein the transgenic mammal comprises a nucleic acid sequence encoding a PDGF A chain and a nucleic acid sequence encoding a PDGF B chain.

8. A method of producing a transgenic mammal capable of expressing an active PDGF molecule in its milk, comprising
introducing into a cell a nucleic acid sequence encoding a PDGF chains operably linked to a promoter which directed expression in mammary epithelial cells; and
allowing the cell to give rise to a transgenic mammal, wherein the transgenic mammal expresses PDGF in its milk and at least 30% of the PDGF is present in the milk is in active form.

9. The method of claim 8, wherein the cell is an oocyte.

10. The method of claim 8, wherein the cell is a somatic cell, and the somatic cell or the nucleus of the somatic cell is introduced into an oocyte.

11. A method of producing a transgenic mammal capable of expressing an active PDGF molecule in its milk, comprising:
introducing into a cell a nucleic acid sequence encoding a PDGF A chain operably linked to a promoter which directs expression in mammary epithelial cells;
introducing into the cell a nucleic acid sequence encoding a PDGF B chain operably linked to a promoter which directs expression in mammary epithelial cells; and
allowing the cell to give rise to a transgenic mammal, wherein the transgenic mammal expresses PDGF in its milk and at least 30% of the PDGF is present in the milk in active form.

12. The method of claim 11, wherein the cell is an oocyte.

13. The method of claim 11, wherein the cell is a somatic cell, and the somatic cell or the nucleus of the somatic cell is introduced into an oocyte.

14. A method of producing a transgenic mammal capable of expressing an active PDGF molecule in its milk, comprising:
providing a cell from a transgenic mammal whose germ and somatic cells comprise a nucleic acid sequence encoding a PDGF-A chain operably linked to a promoter which directs expression in mammary epithelial cells;
introducing into the cell a nucleic acid sequence encoding a PDGF-B chain operably linked to a promoter which directs expression in mammary epithelial cells; and
allowing the cell to give rise to a transgenic mammal, wherein the transgenic mammal expresses PDGF in its milk and at least 30% of the PDGF is present in the milk in active form.

15. The method of claim 14, wherein the cell is an oocyte.

16. The method of claim 14, wherein the cell is a somatic cell, and the somatic cell or the nucleus of the somatic cell is introduced into an oocyte.

17. A milk preparation obtained from a transgenic mammal whose genome contains a nucleic acid sequence encoding at least one PDGF chain operably linked to a promoter which directs expression in mammary epithelial cells, wherein the PDGF chain is expressed in the mammary epithelial cells the transgenic mammal and wherein at least 30% of the PDGF in the milk is present as a dimer.

18. The milk preparation of claim 17, wherein the PDGF chain is the PDGF A chain and at least 30% of the PDGF is present in the milk is as a PDGF-AA homodimer.

19. The milk preparation of claim 17, wherein the PDGF chain is the PDGF B chain and at least 30% of the PDGF is present in the milk is as a PDGF-BB homodimer

20. The milk preparation of claim 17, wherein the genome of the transgenic mammal comprises a nucleic acid sequence encoding a PDGF A chain under the control of a promoter which directs expression in mammary epithelial cells and a nucleic acid sequence encoding a PDGF B chain under the control of a promoter which directs expression in mammary epithelial cells.

21. The milk preparation of claim 20, wherein at least 30% of the PDGF present in the milk is as a PDGF-AB heterodimer.

22. The milk preparation of claim 17, wherein the PDGF is human PDGF.

23. The milk preparation of claim 17, wherein the transgenic mammal is a goat.

24. The milk preparation of claim 17, wherein the milk preparation comprises at least 1 mg/ml PDGF.

25. An isolated nucleic acid comprising a nucleic acid sequence encoding a biologically active PDGF or a homolog thereof operatively linked to a regulatory sequence capable of directing the expression of PDGF in the mammary gland of non-human transgenic mammals.

26. The nucleic acid of claim 25, wherein the nucleic acid sequence encodes a PDGF A chain.

27. The nucleic acid of claim 25, wherein the nucleic acid sequence encodes a PDGF B chain.

28. The nucleic acid of claim 26, wherein the nucleic acid sequence further encodes a PDGF B chain.

29. The nucleic acid of claim 25, wherein the nucleic acid sequence coding for PDGF is mono- or dicistronic.

30. The nucleic acid of claim 25, wherein the nucleic acid sequence is dicistronic.

31. The nucleic acid of claim 25, wherein the nucleic acid comprises the expression cassette BC701 or BC734.
